(19) United States
(12) Patent Application Publication
(10) Pub. No.: US 2026/0345654 A1
(43) Pub. Date: Nov. 5, 2026

(54) 2,8-DIHYDROXYQUINOLINE GLUCURONIDE DERIVATIVES WITH ENHANCED PROPERTIES FOR USE AS ANTICANCER, ANTIVIRAL, ANTIMICROBIAL, AND OTHER THERAPEUTIC APPLICATIONS

(71) Applicant: WESTOL, LLC., Niceville, FL (US)

(72) Inventors: Daniel C. CARTER, Huntsville, AL (US); Gary E. WEST, Niceville, FL (US)

(21) Appl. No.: 19/488,289

(22) PCT Filed: Jun. 3, 2024
(86) PCT No.: PCT/US2024/032216

§ 371 Date: Nov. 26, 2025

Related U.S. Application Data
(60) Provisional application No. 63/505,847, filed on Jun. 2, 2023.

(51) Int. Cl. A61K 31/706; A61P 35/00

(52) U.S. Cl. CPC A61K 31/706; A61P 35/00

(57) ABSTRACT

In accordance with the disclosure, 2,8-Dihydroxyquinoline-beta-D-glucuronide and other related compounds are provided for use in methods of treating cancer and other ailments such as antiviral and antimicrobial methods. One such compound is the intermediate metabolic breakdown product 8-Hydroxy-2-quinolinyl beta-D-glucopyranosiduronic acid (or G2-8HQ). The compound G2-8HQ and other similar compounds are used in compositions and methods for the treatment of cancer and other conditions including antiviral, antimicrobial, and other therapeutic applications. Other hydroxyquinoline compounds are provided for use in treating cancer and other conditions, including 2,8-quinolinediol (2-8HQ), and further compounds including those glucose or carbohydrate derivatives at the hydroxyquinoline substituted at positions 3-7 of the hydroxyquinoline ring. These compounds can be used individually or in combinations with other moieties including other hydroxyquinolines and omega fatty acids, and the therapeutic effectiveness of these compounds can be amplified in the presence of a chelating metal such as copper or other metal chelates.

### CROSS-REFERENCE TO RELATED APPLICATIONS

This application claims the benefit of U.S. Provisional Application Ser. No. 63/505,847 filed Jun. 2, 2023, the disclosure of said application being incorporated herein in its entirety.

### FIELD

The presently disclosed subject matter relates in general to the use of 2,8-Di-hydroxyquinoline glucuronide derivatives, and more specifically to the use of an isolated metabolic breakdown product known as 8-Hydroxy-2-quinolinyl beta-D-glucopyranosiduronic acid (referred to herein as “G2-8HQ” or “W1469”) in compositions and methods for the treatment of cancer and other therapeutic applications including antiviral, antimicrobial, and anti-inflammatory microbial treatments. The invention also relates to the use of another hydroxyquinoline derivative known as 2,8-quinolinediol (2-8HQ) which can also be used in the same manner as G2-8HQ to treat cancers and other ailments. Still other hydroxyquinoline glucuronide derivatives with similar chemistry are provided, including those hydroxyquinoline derivatives with glucose, carbohydrate, or other substitutions at the hydroxyquinoline positions 3-7 on the hydroxyquinoline ring as described herein. These compounds can be used individually, or in complexes with copper or other metals that enhance their therapeutic effectiveness, and/or in combinations with omega fatty acids or other phospholipids.

### ABBREVIATIONS

As used herein, the present disclosure will make reference to the following abbreviations as needed:
8HQ: 8-hydroxyquinoline (W1469p)
CQ: Clioquinol; 5-chloro-7-iodo-8-hydroxyquinoline
GluCQ: Clioquinol glucoconjugate; 5-chloro-7-iodo-8-quinolinyl-β-D-glucopyranoside
Glu8HQ: 8-quinolinyl beta-D-glucopyranoside
G8HQ: 8-quinolinyl beta-D-glucopyranosiduronic acid
G8CQ: 5-chloro-7-iodo-8-quinolinyl beta-D-glucopyranosiduronic acid
G2-8HQ: 8-Hydroxy-2-quinolinyl beta-D-glucopyranosiduronic acid (W1469)
2-8HQ (or 2,8HQ): 2,8-quinolinediol
DHA: docosahexaenoic acid
DMSO: dimethylsulfoxide
PBS: phosphate buffered saline

### BACKGROUND

In the ongoing search to find new ways to effectively treat cancer, one particular family of compounds has been extensively studied over the past few years. This family is known as the 8-hydroxyquinoline pharmacophore family, and in general, there has been some evidence that the use of metal ionophore/chelating compounds from this family may be useful as anti-cancer agents. See, e.g., Toyokuni 1996; Brewer 2000; Theophanides, 2002; Desoize. 2003; and Huang 2005, as identified later herein). Among the most highly studied is the pharmacophore platform centered around 8-hydroxyquinoline (8HQ), a compound discovered in 1895 and applied as an early antimicrobial in the 1940's at the dawn of modern antibiotics. With the advent of modern organic synthetic methods, a modified 8HQ, Clioquinol (CQ), was produced in the 1950's and gained FDA approval for applications globally as an effective antibiotic and anti-diarrhea treatment (Calhoon 2009).

However, in the 1970's Japan reported a serious and tragic side effect associated with the use of CQ in the Japanese population causing much harm and curtailing much of the early research in this area (Tasabaki 1971; Kono, 1971, Meade 1975). In all, many thousands of Japanese were diagnosed with CQ-associated subacute myelo-optic neuropathy which led to this compound being removed from the market in Japan, the United States and many other parts of the world.

Although it was unclear at this time whether this phenomenon was unique to Japan due to genetic or other factors, it appears that these issues arose due to a highly geographically focused abnormality on CQ toxicity. Yet despite the serious safety issue, CQ is still in use in some other parts of the world.

Still, despite these setbacks, there have been additional recent efforts to make use of the ionophores in this general compound family, albeit with mixed results of effectiveness and safety. For example, there have been additional attempts focusing on the synthesis of new 8HQ analogs in an effort to improve safety, solubility, and efficacy in a variety of application areas, again with mixed results. In one case, a drug for human use was developed that was a second generation 8HQ-type drug intended to be a successor to CQ, called PBT2, and this was tried for the treatment of Alzheimer's and Huntington's disease (Cherny 2012). Because of the immense potential and often micromolar or nanomolar activities of 8HQ derivatives for diverse medical indications, CQ and many 8HQ derivatives continue to be explored as new treatments for clinically relevant pathogens (see You 2018; Bednarz-Prashad 1983: Auld 1974; Olaleye 2011; Darby 2010; Tavares 2018; and Bohlmann 2018), including certain attempts dealing with cancer and neurodegenerative diseases (e.g., Schimmer 2012; Ayton 2015; Tavares 2020; Cherny 2012; Lannfelt 2008; Ritchie 2003; Adlard 2008; and Shi 2020). The current state-of-the-art of 8HQ-based pharmaceutical development has been recently and extensively reviewed by Gupta et al., (2021), with FIG. 14 of that reference illustrating the so-far untapped broad range of potentially beneficial biological activity from these compounds, as described further herein. Some exemplary compounds in this family are shown herein in FIG. 15 of this disclosure.

As the previous studies have shown, the function of 8HQ and its derivatives has largely been attributed to their inherent chromophore heavy metal chelating properties, most notably copper (Cu), iron (Fe), and zinc (Zn). In some cases, the function is associated with the direct metal interference, such as in the inhibition of metalloprotease, and in others, the alteration appears to be in transition metal homeostasis or the creation of ROS (reactive oxidation species) such as due to free radical, oxidative stress. More recently, there also have been attempts to use these compounds against COVID 19, and CQs activity in this area appeared to be attributed to blocking viral attachment to ACE receptor required for viral spike protein mediated infection. However, in that case, copper chelation did not appear to be required for the anti-viral function. As a result, investigators in the area of 8HQ compounds continue to search for those compounds that will be effective in treatments for conditions such as cancers, but also can be safely used with limited or no harmful side effects.

Despite the intense studies of 8HQ and its related compounds, there are no previous studies that focus specifically on G2-8HQ having any role in some of the therapeutic effects previously seen with 8HQ, including some works that have incorporated simple glucose or glucuronic acid to the main pharmacophore frame of 8HQ. Among these alternatives, there are only a small number of studies as reflected in a review by Oliveri 2016. Consequently, there are only a handful of works that even refer to research involving the simple O-linked direct frame glucose or glucuronic acid derivatives of 8HQ. Further, most of these studies focused only on glucose modifications directly to the oxygen at position 8 to evaluate a prodrug approach, but such an approach would abrogate the metal binding properties and biological activity without enzymatic activation. These works are summarized below.

In Monson 1991, the authors studied simple 8-hydroxy quinolone-Glucuronide frame derivatives. In these studies, the potential of anti-tumor targeting utilizing the glucuronide derivations of pharmaceuticals using G8HQ as a model system was tested. This work reported certain aspects of glucuronide G8HQ concerning tumor targeting, and the biodistribution of the active aglycone, 8HQ, in tissues of mice at various time points was also studied. However, in their initial animal experiments they found no significant tumor or general tissue partitioning. The authors eventually found that by using this approach, the prior acidification of tumors by the induction of hyperglycemia was required. After acidification, tumor uptake was rapid and significantly increased showing significant tumor targeting and G8HQ activation as summarized in Table 1 on that reference. Tumors shows rapid initial and preferential uptake of G8HQ in first 10 minutes, peaking at 45-60 minutes, but with prolonged retention in tumor and the colon over all other tissues. Because they were measuring the aglycone directly, this work does provide some evidence of the anticipated prodrug cleavage by beta-glucosidase resident in cancer cells and augmented by the preferential uptake by the abundant GLUT receptors on the tumor surface. The study found that G8HQ was metabolically cleared in approximately 3 hours. It is noted that in each case, these were brief experiments over the course of 24 hours after which point the study and animals were terminated so there was no particular follow-up to this specific study.

In a second reference, Yesilagac 2011, the compound 4-iodo, 8-hydroxy beta-D-glucopyranosiduronic acid was studied by incorporating radioactive iodine in order to examine bio distribution and demonstrate the potential for both radiation diagnostics and radiation treatment. Their bio distribution was similar to Monson, but with distinct differences. Clearly, as an iodinated derivative, this compound would not be expected to have the same bio distribution and pharmacokinetic properties as G8HQ. Both Yesilagac, 2011 and Monson 1991 found some evidence for enteropathic recirculation which prolongs the half-life, with Yesilagac finding excretion of the iodinated drug is mainly urinary, and Monson finding that the G8HQ/8HQ drug is largely eliminated at 3 hours post injection. Once again, both studies raised interesting questions about possible use 8HQ derivatives in anti-tumor targeting, but without providing confirmation of long-term results.

The next study was Oliveri 2012 which involved a simple 8-hydroxy quinolone-Glucose and galactose quinoline frame derivatives. While the previous Monson 1991 reference had recognized the overexpression of beta glucosidase in cancer cells as an opportunity to use glucuronides to target cancer cells and simultaneously activate an anti-cancer prodrug; the Oliveri 2012 reference studied similar 8HQ derivatives but with glucose vs. glucuronic acid, and this reference also recognized the potential requirement of copper addition for biological activity/toxicity. In this reference, Oliveri noted the significantly elevated distribution of copper in the blood of cancer patients associated with certain malignant tissues, which had been previously recognized many years ago. See, e.g., Heiger 1926. Many factors point to the enhanced requirement for copper in cancer cells to support the altered metabolic pathway characterized as the Warburg effect (See Vander Heiden 2009).

Indeed, other studies have shown that cancer cells have two copper transporters: CTR1 (influx high-affinity uptake transporter (encoded by the SLC31A1 gene)) and ATP7A (copper exporter-efflux (also knowns as Menkes' protein, a copper-transporting P-type ATPase)).

This is shown for example in Hordyjewska 2014 which states:
“The evidence obtained over the years has shown that cancer cells generally require more copper for their growth and metabolism than resting cells. Therefore agents that affect copper homeostasis are of interest for cancer therapy,” High serum and tissue levels of copper were found in various human tumors including: Hodgkin's lymphoma, leukemia, sarcoma, brain, cervix, lung, breast and liver cancers. It has also been shown that serum copper levels return to normal on remission of the disease or upon removal of the tumor. It has also been demonstrated that copper is required for angiogenic process.”

Moreover, this was further elaborated in a more recent reference, Oliveri 2022, which stated that:
“[S]everal studies have reported higher levels of Cu in a variety of malignancies compared to normal tissues. Cu accumulation has been associated with enhanced proliferation and growth, angiogenesis, and metastasis. It is evident that the dyshomeostasis of Cu plays a prominent role in cancer, although researchers debate if it is a cause or a consequence of tumorigenesis. In particular, Cu levels of both serum and tumor tissues have been found significantly altered in patients suffering from different cancers such as breast, thyroid, cervical, ovarian, lung, pancreatic, prostate, gastric, oral, bladder cancers (Basu et al., 2013; Ding et al., 2015; Pavithra et al., 2015; Baltaci et al., 2017; Stepien et al., 2017; Zhang and Yang, 2018; Chen et al., 2019; Aubert et al., 2020; Saleh et al., 2020; Michniewicz et al., 2021). There is some evidence that Cu could have a role in the etiology, severity, and progression of cancer disease (Ishida et al., 2013; Shanbhag et al., 2021).”

Further, recognition of the selective disruption of copper involved in cancer cellular signaling has recently been highlighted in top journals as a new approach for the development of more effective therapeutics (Kahlson 2022).

As a result of these and other studies, it appears that the 8HQs in combination with copper could play an important role in cancer treatment. For example, numerous studies have shown that there are two or more potential therapeutic pathways of copper binding therapeutics, namely those which are ionophores and facilitate the transfer and accumulation of copper with the target cell, those which are copper chelators, which bind and limit the copper dependent oncogenic pathways, and those chelated forms which create copper redox centers promoting oxidative stress and the induction of apoptosis For CQ, as described above, the presence of copper has been similarly determined to be an enhancer or prerequisite of the anti-cancer activity (Zhai 2010), and confirmed again by Oliveri 2012. The ionophore-induced anticancer activity of CQ has been explored in Yu 2009 wherein in the special case of Zn and a prostate cancer cell line, the mechanism was determined to be specific transport and deposition of the transition metal in the cell lysosome where it disrupts normal function and induces apoptosis. However, the majority of studies, point to copper as the important trace metal for the anticancer activity, not zinc.

In other studies, the anticancer activity of CQ and its analogs have also been reported to increase synergistically with the addition of omega fatty acids such as docosahexaenoic acid (DHA) (see Ding 2006), a known lipid with anti-carcinogenic properties (see Bougnoux 1999; Begin 1986; and Ding 2004). These findings are consistent with the observation of elevated levels of omega 3 and omega 6 fatty acid forms (phospholipids) in the serum of goats with higher levels of anticancer activity as shown below. While the anticancer activity of DHA has been attributed to lipid peroxidation, the mechanism of enhanced activity (greater than the sum of the cytotoxicity of each agent) remains undetermined, although it strongly suggests that ROS is the major source of the anti-cancer activity.

However, this synergy has been observed by others, including Baumgartner 2004 and Germain 1998), which implies that the lipid peroxidation and reactive oxygen species (ROS) may be enhanced by the presence of the increased intracellular copper bound drug concentration. Of further importance to the present ionophore discussed herein, an additional potential synergy of the addition of DHA relates to DHA's upregulation and expression of Beta-glucosidases required to activate certain prodrug forms.

Of further relevance to this field, Oliveri 2012 designed O-linked glucose adducts to the 8-hydroxy position of both 8HQ and CQ, and the resulting pro-drug form abrogated the copper binding and eliminated the anti-cancer activity until re-activated intracellularly by beta-glucosidase. The authors of this reference similarly reasoned that the glucose adduct would improve solubility, target cancer cells to a higher degree of specificity due to an overabundance of glucose transport receptors (GLUT) and similarly potentially enhancing transport across the blood brain barrier where these receptors are also abundant. The anaerobic metabolism and high dependence on glucose that characterizes most cancers is a phenomenon known as the “Warburg Effect” (see Vander Heiden 2009). They further reasoned that the glucose adduct functions as an inactive prodrug to be converted to the active 8HQ form by the hydrolysis of the glycosidic bond by beta-glucosidases in the cytoplasm of cancer cells.

To compare the anti-proliferative properties of these glucoconjugates, Oliveri 2012 further examined their performance against three Human cell lines: A2780 (ovary, adenocarcinoma), A549 (lung, carcinoma) and MDA-MB-231 (breast, carcinoma). The authors demonstrated that the addition of Cu⁺² to the media significantly increased the cytotoxicity of 8HQ, CQ and their respective 8-hydroxy glucose modifications, and provided further evidence that glucose adducts to the 8-hydroxyl position (prodrug) are activated intracellularly in the cytoplasm by beta-glucosidases, releasing the metal binding and potent anti-proliferative activity of 8HQ. In a later study, O-linked chlorinated derivative compounds 32 and 33 showed the most cytotoxicity in the presence of copper.

As such, these experiments are supportive of the earlier results reported in Monson 1991 relating to a glucuronide derivative at the 8-hydroxyl (G8HQ). In those cases, IC₅₀s for the various compounds were enhanced in the presence of 20 μM Cu⁺², increasing from 9× for GluCQ to 18× for Glu8HQ. In addition, although not recognized in Oliveri 2012, the elevated copper concentrations found in the blood of many cancer patients has been determined by others to be approximately 20 μM, thus leading to this selection of concentration in their studies. The Oliveri results for synthetic Glucose derivatives of 8-hydroxyquinol (X=Z=H) (Glu8HQ) and 5-chloro-7-iodo-8-hydroxyquinoline (X=I, Z=Cl) (GluCQ) are illustrated and demonstrate that glucose modification to 8HQ (Glu8HQ) appears to be superior to CQ (GluCQ).

Over the years, there have been a number of synthesized compounds in this area studied for their antiproliferative properties. Unlike the glucose modifications (compounds 31-34), the galactose modifications (compounds 35-40) were not able to be cleaved by the beta glucosidase, and thus this resulted in a loss of cytotoxicity (see Oliveri 2016). Moreover, in the same review, it was noted that trehalose derivatives at position 2 incorporated by either an amide or amine moiety, as well as linked polysaccharides at a variety of frame positions, show low antiproliferative activity, and the inclusion of copper ions did not affect their activity. The authors proposed that these derivatives were not recognized by the GLUTs or were too polar to pass through the cellular membrane.

As a result, although the extensive prior studies of 8HQ and its relating compounds have provided some promise in in targeting tumor cells and treating cancer, it has still been difficult to isolate specific compounds that show the required high level of anti-tumor activity, yet which can also be made into stable and effective anti-cancer compositions and methods that are safe and can be administered with little or no unwanted side effects.

### SUMMARY

In accordance with the present disclosure, compositions and methods are provided for treating cancer and other conditions that comprise as the active ingredient 2,8-Di-hydroxyquinoline glucuronide derivatives, and more specifically the metabolic byproduct 8-Hydroxy-2-quinolinyl beta-D-glucopyranosiduronic acid (referred to herein as “G2-8HQ” and “W1469”). The compound 8-Hydroxy-2-quinolinyl beta-D-glucopyranosiduronic acid (which is also known as 2,8-Dihydroxyquinoline-beta-D-glucuronide) has the formula as shown below:

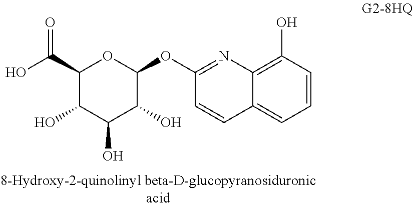 [SMILES: O=C(O)[C@H]1O[C@@H](Oc2ccc3cccc(O)c3n2)[C@H](O)[C@@H](O)[C@@H]1O]

The invention also relates to the use of G2-8HQ, its stereoisomers and congeners, and other compounds with similar chemistry, such as those glucose or carbohydrate derivatives at positions 3-7 of the hydroxyquinoline ring. These compounds include those O-linked glucose or carbohydrate derivatives wherein the substitution is at the hydroxyquinoline positions 3-7 instead of the substitution at position 2 as in G2-8HQ. These compounds can be used individually or in combinations with omega fatty acids, and the therapeutic effectiveness of these compounds can be amplified in the presence of copper.

In addition, in accordance with the present invention, compositions and methods are provided for treating cancer and other conditions that comprise using as the active ingredient 2,8-quinolinediol (referred to herein as “2-8HQ”). The formula for 2,8-quinolinediol is as shown below:

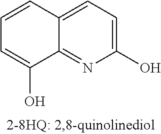 [SMILES: Oc1ccc2cccc(O)c2n1]

This diol is also known as 2,8-dihydroxyquinoline (2-8HQ) and it is contemplated that this compound or its natural enantiomeric tautomers can also be utilized in accordance with the present invention. As set forth below, both G2-8HQ and 2-8HQ may be used in compositions and methods for treating cancers and a number of ailments as described further herein.

In accordance with the present disclosure, an isolated compound is provided for use in treating cancer and other conditions, and that compound is 8-Hydroxy-2-quinolinyl beta-D-glucopyranosiduronic acid (G2-8HQ) as shown above. The compound G2-8HQ is an obscure and long overlooked metabolic product found in the serum of many herbivores including ungulates such as goats, and which had never been considered or studied for therapeutic purposes. However, as set forth in the present disclosure and as described herein, this compound may be isolated or synthesized for use in treating cancers and other conditions such as viral or microbial infections, asthma, neurodegenerative disease, and/or inflammation and inflammatory conditions in various ailments.

In one exemplary embodiment, a pharmaceutical composition is provided which comprises 8-Hydroxy-2-quinolinyl beta-D-glucopyranosiduronic acid (G2-8HQ) and a pharmaceutically acceptable vehicle, excipient, or carrier. In certain embodiments, the 8-Hydroxy-2-quinolinyl beta-D-glucopyranosiduronic acid may be complexed with a chelating metal such as copper and other metals such zinc, iron, cobalt, rhodium, and platinum, among others that would be known by those of ordinary skill in the art to be able to form complexes with the hydroxyquinolines of the invention. As indicated herein, the complexing of G2-8HQ with copper or other chelating metals can greatly enhance the effect of the active compound in treating cancer and reducing cancerous tumors.

In other exemplary embodiments, the G2-8HQ, either alone or in the form of a copper or other metal complex as described above, may be further combined with a suitable fatty acid, such as an omega fatty acid. Such fatty acids may be omega-3 fatty acids such as alpha-linoleic acid (ALA), eicosapentaenoic acid (EPA) and docosahexaenoic acid (DHA), or omega-6 fatty acids such as linoleic acid (LA), arachidonic acid (AA), docosapentaenoic acid (DPA), or any combination thereof.

In still other exemplary embodiments, G2-8HQ may also be used along with another hydroxyquinoline, for example, 8-hydroxyquinoline (8HQ), and in certain embodiments, a pharmaceutical composition is provided that comprises 8-Hydroxy-2-quinolinyl beta-D-glucopyranosiduronic acid (G2-8HQ) and a compound selected from the group consisting of 8HQ, an omega-3 fatty acid, and an omega-6 fatty acid and a pharmaceutically acceptable vehicle, excipient, or carrier. Examples of the omega-3 fatty acids are alpha-linoleic acid (ALA), eicosapentaenoic acid (EPA) and docosahexaenoic acid (DHA), and examples of the omega-6 fatty acid include linoleic acid (LA), arachidonic acid (AA), docosapentaenoic acid (DPA), or any combination thereof.

As set forth in more detail herein, the active ingredients of the pharmaceutical compositions of the present disclosure for example 8-Hydroxy-2-quinolinyl beta-D-glucopyranosiduronic acid (G2-8HQ) and as 2,8-dihydroxyquinoline (2-8HQ), will be utilized in treatments for cancer and other ailments including viral and other microbial infections, asthma, neurodegeneration, and inflammation. In such treatments, and effective amount of these active ingredients will be administered to a patient in need thereof, and as described herein, the effective amount is variable and will be determined by the doctor or other clinician based on the circumstances of the ailment and the condition of the patient. Accordingly, there may be a wide range of effective concentrations for the active ingredients, as reflected herein. In general, the concentration of the hydroxyquinoline derivatives of the invention including 8-Hydroxy-2-quinolinyl beta-D-glucopyranosiduronic acid (G2-8HQ) and as 2,8-dihydroxyquinoline (2-8HQ) will range from about 100 uM to about 500 mM, and in other exemplary embodiments, this concentration may be in the range of about 300 μM to about 150 mM, from about 1 mM to about 50 mM, or from 1 mM to about 25 mM. In addition, as set forth above, the active ingredients described above may be supplemented by other compounds including an additional hydroxyquinoline, such as 8-hydroxyquinoline (8HQ) and/or an omega fatty acid, and in all cases, results may be enhanced when the active ingredients are administered in the presence of a chelating metal such as copper.

In an exemplary embodiment of the disclosure, a method for treating cancer is provided that comprises administering an effective amount of an active hydroxyquinoline derivative ingredient such as 8-Hydroxy-2-quinolinyl beta-D-glucopyranosiduronic acid (G2-8HQ) or 2,8-dihydroxyquinoline (2-8HQ), and these compounds may be administered in the form of a pharmaceutical composition that includes a pharmaceutically acceptable vehicle, carrier, or excipient as would be readily known by those or ordinary skill in the art. The active ingredients may be administered in any suitable form as would be determined by the doctor or clinician based on the circumstances and condition of the patient. In exemplary embodiments, the administration may be any of a variety of suitable forms, including oral or parenteral administration, injection, intravenous (IV) administration, a suppository, topical administration, and/or in the form of a spray mist. In addition, the hydroxyquinoline derivatives as described herein may be administered in the presence of a chelating metal such as copper or other suitable metal such as zinc, iron, cobalt, rhodium, platinum, etc. As is further described herein, complexing the active ingredients with a suitable chelating metal such as copper has been observed to enhance the therapeutic abilities of the active compounds as described herein.

In additional exemplary embodiments, the hydroxyquinoline derivatives as described herein may be administered in the presence of another hydroxyquinoline such as 8-hydroxyquinoline (8HQ) and/or an omega fatty acid such as an omega-3 fatty acid or an omega-6 fatty acid. In certain embodiments, the omega-3 fatty acid is selected from the group consisting of alpha-linoleic acid (ALA), eicosapentaenoic acid (EPA), and docosahexaenoic acid (DHA), land the omega-6 fatty acid is selected from the group consisting of linoleic acid (LA), arachidonic acid (AA), and docosapentaenoic acid (DPA), or any combination thereof.

In other embodiments of the present invention, the hydroxyquinoline derivatives as described herein including 8-Hydroxy-2-quinolinyl beta-D-glucopyranosiduronic acid (G2-8HQ) and as 2,8-dihydroxyquinoline (2-8HQ) may be used in methods of treating a condition selected from the group consisting of viral infection, a microbial infection, asthma, neurodegeneration, and inflammation, Such methods will comprise administering an effective amount of the composition of claim 1 to a subject in need thereof. As indicated herein, the effectiveness of the active hydroxyquinoline ingredients may be enhanced by administering these agents in the presence of a chelating metal such as copper, zinc, iron, cobalt, rhodium, and platinum, or any other suitable chelating metal that would be well known to those of ordinary skill in the art. The active hydroxyquinoline ingredients may also be administered along with other compounds such as 8-hydroxyquinoline (8HQ) and/or an omega fatty acid such as an omega-3 fatty acid or an omega-6 fatty acid.

In one exemplary embodiment of the invention, a pharmaceutical composition is provided for use in treating cancer and other ailments that comprises 8-Hydroxy-2-quinolinyl beta-D-glucopyranosiduronic acid (G2-8HQ) and a compound selected from the group consisting of 8HQ, an omega-3 fatty acid, and an omega-6 fatty acid and a pharmaceutically acceptable vehicle, excipient, or carrier. In such compositions, the omega-3 fatty acid may be alpha-linoleic acid (ALA), eicosapentaenoic acid (EPA), or docosahexaenoic acid (DHA), and the omega-6 fatty acid may be linoleic acid (LA), arachidonic acid (AA), docosapentaenoic acid (DPA), or any combination thereof.

In another exemplary embodiment of the invention, a pharmaceutical composition is provided that comprises 8-Hydroxy-2-quinolinyl beta-D-glucopyranosiduronic acid or 2,8-dihydroxyquinoline (2-8HQ) and a chelating metal. In certain embodiments the chelating metal is copper, and in other embodiments, the metal may be any suitable metal including but not limited to zinc, iron, cobalt, rhodium, and platinum. In general, the concentration of the 8-Hydroxy-2-quinolinyl beta-D-glucopyranosiduronic acid (G2-8HQ) or 2,8-dihydroxyquinoline (2-8HQ) used in the pharmaceutical compositions of the invention will range from about 100 μM to about 500 mM, and in other exemplary embodiments, this concentration may be in the range of about 300 μM to about 150 mM, from about 1 mM to about 50 mM, or from 1 mM to about 25 mM. In addition, as set forth above, the active ingredients described above may be supplemented by other compounds including an additional hydroxyquinoline, such as 8-hydroxyquinoline (8HQ) and/or an omega fatty acid.

In another embodiment of the present invention, a method of treating cancer comprising administering an effective amount of the pharmaceutical composition comprising hydroxy-2-quinolinyl beta-D-glucopyranosiduronic acid or 2,8-dihydroxyquinoline (2-8HQ) and a chelating metal to a human or animal patient in need thereof. Such administration may be in a variety of suitable forms including oral, rectal, parenteral, injection, intravenous (IV), suppository, topical, and spray/mist. This pharmaceutical composition may also be administered in combination with another hydroxyquinoline such as 8HQ and/or an omega fatty acid.

In an exemplary embodiment of the present invention, a method is provided for treating a variety of ailments such as a viral infection, a microbial infection, asthma, neurodegeneration, or an inflammatory condition that comprises administering an effective amount of the pharmaceutical composition comprising hydroxy-2-quinolinyl beta-D-glucopyranosiduronic acid or 2,8-dihydroxyquinoline (2-8HQ) and a chelating metal to a human or animal patient in need thereof. Such administration may be in a variety of suitable forms including oral, rectal, parenteral, injection, intravenous (IV), suppository, topical, and spray/mist. This pharmaceutical composition may also be administered in combination with another hydroxyquinoline such as 8HQ and/or an omega fatty acid.

As described herein, one suitable hydroxyquinoline compound for use in treating cancer and other ailments such as viral and bacterial infections, asthma, neurodegeneration, and inflammation is 2,8-quinolinediol (2-8HQ). The compound 2,8-quinolinediol is another metabolic breakdown product and is believed to be the result of intracellular conversion of G2-8HQ by keto-enol chemistry at neutral to low pH and/or the β glucosidase hydrolysis reaction wherein 2-8HQ is released. 2-8HQ is also an active Cu+2/Zn+2 chelator and thus may be complexed with a chelating metal such as copper, zinc, or a number of other suitable metals that would be well known in the art. In certain embodiments, 2,8-quinolinediol (2-8HQ). is used in the form of a pharmaceutical composition comprising 2,8-quinolinediol and a pharmaceutically acceptable vehicle, excipient, or carrier, and this pharmaceutical composition may also include a metal such as copper, zinc, or other suitable metals. Still further, this pharmaceutical composition may also comprise an omega fatty acid and or an additional hydroxyquinoline such as 8-hydroxyquinoline (8HQ).

In additional embodiments, the present invention comprises a pharmaceutical composition for use in treating cancer and other ailments where the pharmaceutical composition compromises 2,8-quinolinediol (2-8HQ) and a compound selected from the group consisting of 8HQ, an omega-3 fatty acid, and an omega-6 fatty acid and a pharmaceutically acceptable vehicle, excipient, or carrier. This composition may also include a metal that will form a chelate complex with the active ingredient, and the metal will be any suitable metal capable of forming such complexes, including but not limited to copper, zinc, iron, cobalt, rhodium, and platinum.

In accordance with the present disclosure, a method for treating cancer and other ailments is provided comprising administering a pharmaceutical composition comprising 2,8-quinolinediol (2-8HQ) and a compound selected from the group consisting of 8HQ, an omega-3 fatty acid, and an omega-6 fatty acid, along with a pharmaceutically acceptable vehicle, excipient, or carrier. This composition may also include a metal that will form a chelate complex with the active ingredient, and the metal will be any suitable metal capable of forming such complexes, including but not limited to copper, zinc, iron, cobalt, rhodium, and platinum. The metals may also be in their ionic form, as would be recognized by one of ordinary skill in the art. In exemplary embodiments of the present method, treatment may be carried out in any suitable form, such as oral, rectal, parenteral, injection, intravenous (IV), suppository, topical, and a spray of mist, such as an inhaler. In a further embodiment of this method, the active ingredients may also be administered with an omega fatty acid such as alpha-linoleic acid (ALA), eicosapentaenoic acid (EPA), docosahexaenoic acid (DHA), linoleic acid (LA), arachidonic acid (AA), and/or docosapentaenoic acid (DPA), or any combinations thereof.

In another embodiment of the present disclosure, a method for treating a condition selected from the group consisting of a viral infection, a microbial infection, a parasitic infection, asthma, neurodegeneration, and inflammation is provided that comprises administering a pharmaceutical composition comprising 2,8-quinolinediol (2-8HQ) and a compound selected from the group consisting of 8HQ, an omega-3 fatty acid, and an omega-6 fatty acid, along with a pharmaceutically acceptable vehicle, excipient, or carrier. This composition may also include a metal that will form a chelate complex with the active ingredient, and the metal will be any suitable metal capable of forming such complexes, including but not limited to copper, zinc, iron, cobalt, rhodium, and platinum. The metals may also be in their ionic form, as would be recognized by one of ordinary skill in the art. In exemplary embodiments of the present method, treatment may be carried out in any suitable form, such as oral, rectal, parenteral, injection, intravenous (IV), suppository, topical, and a spray of mist, such as an inhaler. In a further embodiment of this method, the active ingredients may also be administered with an omega fatty acid such as alpha-linoleic acid (ALA), eicosapentaenoic acid (EPA), docosahexaenoic acid (DHA), linoleic acid (LA), arachidonic acid (AA), and/or and\docosapentaenoic acid (DPA), or any combinations thereof.

In further embodiments of the invention, a pharmaceutical composition complex is provided for use in treating a condition selected from the group consisting of cancer, a viral infection, a microbial infection, asthma, neurodegeneration, and inflammation, said composition comprising 8-Hydroxy-2-quinolinyl beta-D-glucopyranosiduronic acid (G2-8HQ) or 2,8-quinolinediol (2-8HQ) and a chelating metal. The chelating metal may be any suitable metal such as copper, zinc, iron, cobalt, rhodium, and platinum.

In another exemplary embodiment of the invention, a pharmaceutical composition is provided that comprises a glucuronic acid substituted 2,8-dihydroxy quinolone and a pharmaceutically acceptable vehicle, carrier, or excipient. Such substitutions may take advantage of the unique properties of the hydroxyquinoline derivatives of the invention such as G2-8HQ, but also may include the positioning of additional unique glucose or carbohydrate derivatives at the 8HQ positions, 2-7. Such compounds have the desired solubility and targeting and do not have metabolic activation and thus do not act as prodrugs. In exemplary embodiments of the invention, pharmaceutical compositions are provided wherein the glucuronic acid substituted 2,8-dihydroxy quinolone is selected from the group consisting of 8-Hydroxy-2-quinolinyl beta-D-glucopyranosiduronic acid, 8-Hydroxy-3-quinolinyl beta-D-glucopyranosiduronic acid, 8-Hydroxy-4-quinolinyl beta-D-glucopyranosiduronic acid, 8-Hydroxy-5-quinolinyl beta-D-glucopyranosiduronic acid, 8-Hydroxy-6-quinolinyl beta-D-glucopyranosiduronic acid, 8-Hydroxy-7-quinolinyl beta-D-glucopyranosiduronic acid, 2-beta-D-glucopyranosiduronic acid-5-chloro-7-iodo-8-hydroxyquinoline; 8-Hydroxy-2-quinolinyl beta-D-glucopyranoside, 8-Hydroxy-3-quinolinyl beta-D-glucopyranoside. 8-Hydroxy-4-quinolinyl beta-D-glucopyranoside, 8-Hydroxy-5-quinolinyl beta-D-glucopyranoside, 8-Hydroxy-6-quinolinyl beta-D-glucopyranoside, 8-Hydroxy-7-quinolinyl beta-D-glucopyranoside, and 2-beta-D-glucopyranoside-5-chloro-7-iodo-8-hydroxyquinoline. As indicated above, these compounds and the pharmaceutical compositions containing said compounds may also be used in accordance with the invention in treating a variety of conditions including cancer, viral and microbial infections, asthma, neurodegeneration, and inflammation.

With regard to the metabolic aspects of G2-8HQ, it is the case that ungulates (and birds) ingest various plant and seed dietary components which contain 8HQ which is further broken down by the specialized gut microbiome of ungulates to yield the “bacteria” metabolic byproduct 2,8HQ. The compound 2,8HQ is absorbed into the blood stream (most likely by albumin) where glucuronidation in the liver takes place as a major metabolic detoxification system in mammals. This process uses UDP-glucuronosyltransferase to create the soluble metabolic product glucuronide 8-Hydroxy-2-quinolinyl beta-D-glucopyranosiduronic acid (G2-8HQ) of the present invention.

Further, 2,8HQ which is otherwise insoluble in aqueous systems, is most likely solubilized by albumin and highly bound, and is then off loaded by albumin in the liver to create G2,8HQ which is now soluble and freely circulating. In light of this, one possible embodiment of the invention would be to administer 2,8HQ (a very inexpensive precursor) either orally, or by precomplexation with albumin as an alternate treatment approach, where the desired active targeting metabolite is created by glucuronidation in the liver creating the glucuronide derivative G2-8HQ.

In another embodiment of the present invention, a glucopyranosiduronic acid such as G2-8HQ is provided that is substituted with a different sugar at the 2-position, and this compound may be used in the form of a pharmaceutically acceptable composition that also contains a pharmaceutically acceptable vehicle, excipient, or carrier. In certain embodiments, the different sugar at the 2-position is selected from the group consisting of galactose, glucose, fructose, sucrose, and trihalose.

In a further exemplary embodiment, a pharmaceutical composition is provided that comprising 8-Hydroxy-2-quinolinyl beta-D-glucopyranosiduronic acid (G2-8HQ) modified so that an O-linked sugar is at a position other than at position 8, and a pharmaceutically acceptable vehicle, excipient, or carrier. Still further, a pharmaceutical composition is provided that comprises a glucuronic acid substituted 2,8-dihydroxy quinolone and a pharmaceutically acceptable vehicle, excipient, or carrier. In certain exemplary embodiments, the glucuronic acid derivative is 8-Hydroxy-2-quinolinyl beta-D-glucopyranosiduronic acid, and in other embodiments, the glucuronic acid derivative is 8-Hydroxy-quinolinyl beta-D-glucopyranosiduronic acid. These compounds may also be utilized in the form of a complex with a suitable metal such as copper or other metals, and these compositions will also be useful in accordance with the methods of treatment as described herein.

The hydroxyquinolines of the invention as described herein may be obtained by suitable synthesis methods as described in previous articles relating to the research on these compounds as referred to herein. In addition, the compounds of the invention that are metabolic products can be isolated from the serum of ungulates such as goats and numerous other hooved mammals, using methods of separation and isolation known in the art.

As indicated herein, the pharmaceutical compositions containing G2-8HQ, 2-8HQ and the other hydroxyquinoline compounds as described in this disclosure may be used in methods of treating cancer and the other ailments as described herein. These compounds and the compositions containing them may also be used along with a metal to form chelate complexes that enhance the therapeutic effect of these compounds. These pharmaceutical compositions may also contain phospholipids such as an omega fatty acid.

In accordance with the present invention, compositions and methods are provided for treating cancer and other conditions that comprise as the active ingredient 2,8-Di-hydroxyquinoline glucuronide derivatives, and more specifically the metabolic byproduct 8-Hydroxy-2-quinolinyl beta-D-glucopyranosiduronic acid (referred to herein as “G2-8HQ” and “W1469”). The 8-Hydroxyquinolines in general represent a family of molecules highly studied and applied in the field of inorganic and bioinorganic chemistry. They are bi and tridentate metal-chelating compounds known to exhibit a spectrum of potent biological properties which include anti-viral, antimicrobial and anti-cancer activities. In accordance with the current disclosure, the isolation, identification, synthesis, and characterization of several endogenous small molecules in this family for their use in methods of treating cancer. As indicated below, certain of these small molecules of the invention identified from the serum plasma fraction of goats have been shown to exhibit extraordinary in vitro and in vivo activity against a large number of cancers, microbes (e.g., mycobacteria tuberculosis, Cryptococcus) and viruses (e.g., HIV, rotavirus, SARS, COVID 19, etc.)

In exemplary embodiments in accordance with the invention, two of the 8-hydroxyquinols identified with the potential for use in anti-cancer and other therapeutic treatments are 8-hydroxy-2-quinolinyl beta-D-glucopyranosiduronic acid (identified here as G2-8HQ), a metabolic derivative of 8-hydroxyquinoline (8HQ), and 2,8-quinolinediol (identified herein as 2-8HQ). The activities of these compounds reflected in the present studies are described herein.

In one case, the anti-proliferative activity of these quinols against six cancer cell lines was determined where IC₅₀s ranged from 0.2 μM to >500 uM and found after treatment with G2-8HQ. In certain tests, the anti-proliferative activity was seen to be highly dependent on the presence of copper (II). For example, the IC₅₀ for G2-8HQ against the RL non-Hodgkin's lymphoma cell line in the presence of copper increased by more than 1000-fold (from >500 uM-Cu⁺² to IC₅₀ 0.468 μM+Cu⁺²), whereas the same additional presence of copper only increased the activity of 8HQ by 9-fold (IC₅₀ 0.06 μM+Cu⁺²). As a result, in one exemplary embodiment of the present invention, G2-8HQ is utilized along with copper or other metals so as to maximize its ability to be used as an anti-cancer and an anti-microbial agent By comparison with G8CQ, G8HQ, 8HQ, and CQ, in the absence of copper, G2-8HQ is the only drug candidate that shows an absence of cytotoxicity (no measurable IC50 in standard drug concentration ranges). Thus, this evidence that the use of G2-8HQ in treating cancer and other conditions will have a greater safety margin (Therapeutic Index) than any previously reported pro-drug glucose or glucuronide derivatives of 8HQ and its congeners, e.g., clioquinol, yet G2-8HQ exhibits comparable or superior anti-proliferative activity than the prior compounds. Significantly lower toxicity to non-malignant tissues may allow for more extended treatment protocols thereby increasing the likelihood of a favorable clinical outcome.

Additionally, unlike previous pro-drug glucose or glucuronide derivatives which require enzymatic activation via β-glucosidase to release the active aglycone form, beta-glucosidase is not required for the activity of G2-8HQ. Further, the G2-8HQ complex with copper or other metals remains uncharacteristically highly soluble in aqueous solutions, which further its effectiveness and safety as an anti-cancer, anti-microbial, anti-viral, and anti-inflammatory composition.

As indicated above, the compound G2-8HQ is provided for use as an anti-cancer, anti-viral, anti-bacterial, anti-parasitic, anti-fungal agent with enhanced and highly desirable efficacy, but also enhanced safety and clinical administrative characteristics. G2-8HQ represents the first direct ring modified o-linked glucose or glucuronic acid (any sugar) at the 2 position of G2-8HQ to be used in any therapeutic platform. Indeed, in previous studies, G2-8HQ was merely noted as one of many metabolic products found in the serum and tissues of ungulates and birds presumably of dietary and gut microbial origin, and thus there has been no focus on this compound for any therapeutic properties.

As described herein, G2-8HQ has a unique desirable solubility and prodrug-like properties with the further ability to be used in cancer applications to provide improved tumor partitioning. As shown herein, G2-8HQ has powerful anticancer activity against all cell lines tested and further shows the biological activity is further enhanced through copper or other metal chelation. As shown herein, in an exemplary embodiment of the invention, G2-8HQ can be utilized by chelating this compound with copper or other metals such as Zinc (Zn), Iron (Fe), Cobalt (Co), Rhodium (Rh), Platinum (Pt) and others as described further herein.

The unique properties of G2-8HQ as evidenced by the present disclosure make it clear that this compound and compositions comprising this compound can be very useful in methods for treating cancer and a variety of other conditions such as inflammation and viral or bacterial infections. In addition to G2-8HQ, other compounds employed in the present disclosure include those quinoline compounds that add additional unique glucose or carbohydrate derivatives at the positions 2-7 of 8HQ, which will have similar properties to G2-8HQ wherein the glucose or other sugar is at the 8 position. Such compounds also have the desired solubility and targeting, and also do not have the requirement of metabolic activation (i.e., they are not prodrugs). The use of the compounds of the present invention individually or in combination with copper, other metals, and/or omega fatty acids as described herein are thus useful in compositions, methods, and uses related to anti-cancer, anti-inflammatory, anti-viral, and anti-microbial treatments. Further, in accordance with the invention, other compounds are provided in which have a substitution of other sugars at the 2 position (such as galactose, glucose, fructose, sucrose, trihalose, etc.) and any congeners of such sugar-2-8HQ forms thereof are also provided in accordance with the invention.

As also indicated herein, the biological basis for anticancer and antiviral activity present in the sera of selected goats has been determined by assessing two hydroxyl quinoline compounds, 8-hydroxyquinoline (8HQ) and 8-Hydroxy-2-quinolinyl beta-D-glucopyranosiduronic acid (G2-8HQ). Both of these were found at significantly higher concentration, namely 10× and 21× respectively, in the sera of the positive animals as compared to sera from an animal where the activity was considered absent. Although 8HQ has been previously known for its possible anticancer effect, there has been no previous focus on G2-8HQ despite extensive studies of the 8HQ family over the past 20 years. In addition to the two quinols, a selection of phospholipids comprising mainly omega 3 and omega 6 polyunsaturated fatty acids with pre-characterized anti-cancer and immune modulatory properties including anti-inflammatory properties, were also shown to be present at concentrations up to >700× higher in the positive animal (Table 1B). Based on the testing, it was determined that 8-Hydroxy-2-quinolinyl beta-D-glucopyranosiduronic acid (G2-8HQ), either alone or with additional compounds such as omega fatty acids, or in combination with chelating metals such as copper, could be used effectively as an anti-cancer and anti-microbial agent, as described herein.

In light of the foregoing, obtaining and using G2-8HQ and 2-8HQ provide distinct advantages over the prior art attempted uses of 8HQ in the treatment of cancer and other ailments. In the present invention, the adaptation of the 2,8HQ as the active copper chelating quinolone can potentially overcome the disadvantage of poor aqueous solubility, which is counterintuitive based on the additional hydroxyl at position 2. The 2-hydroxy, however has the unique property among all 8-hydroxy quinolone frame derivatives in its inherent keto-enol chemistry. Prior testing of the 8HQs has focused on prodrug forms where the release of the aglycone drug form must be enzymatically accomplished by beta-glucosidase.

However, this is another problem that is overcome by the present disclosure. In the case of G2-8HQ of the present invention which is chemically stable at neutral or physiological pH in the plasma, the reactive chemistry/instability of the glucuronide at position 2 provides a potentially advantageous intracellular pathway at acidic cytosolic pH, to release or “kick-off” the sugar moiety.

Further, another distinction over the prior art such as reflected in the Monson and Oliveri articles referred to herein is that under appropriate conditions, G2-8HQ can chelate copper without enzymatic or keto-enol hydrolysis. Under these conditions, the high standard deviations observed in the present results of in vitro screening as described herein disappears, once again confirming that the G2-8HQ antiproliferative activity makes particularly good use of copper when added, and that the copper complex represents the activated form. In addition, G2-8HQ in complex with copper can be administered with the glucuronic acid/glucose-based targeting intact, unlike any of the prior art described by Monson and Oliveri. Even further, unlike previous hydrolyzed complexes and activated drugs of Glu8HQ and G8HQ, G2-8HQ remains highly aqueous soluble.

As described herein, none of the extensive 8HQ studies in the prior art examined the special O-linked chemistry of the 8-hydroxyquinoline frame as embodied in the present invention. And indeed there have been no reported studies regarding any isolation of antiproliferative or other potential therapeutic studies of the 2,8HQ parent compound or G2-8HQ. As also set forth herein, the hydroxyquinoline compounds that are useful in the present invention may include others involving the same keto-enol chemistry that can be applied to a variety of sugars, as well as congeners of the 8-hydroxy frame, including simple congeners of the main frame such as halide methyl, alcohol, carboxylic acids, etc., and these are provided by virtue of the present invention as well.

Still other derivatives of hydroxyquinolines are provided in the present invention, and these have also shown properties indicative of those described herein for G2-8HQ.

### BRIEF DESCRIPTION OF THE DRAWING FIGURES

FIG. 1. Structure of G2-8HQ: 8-Hydroxy-2-quinolinyl beta-D-glucopyranosiduronic acid.

FIG. 2. Cytotoxicity of WT1469 (G2-8HQ) against A549 breast cancer cell line in the presence of Cu⁺² ions. Note the wide standard deviation at 1 and 0.5 μM indicating that in some % of the screens all the cancer cells were killed at these concentrations. The reason for the large standard deviations for W1469+Cu is discussed vide infra.

FIG. 3. Cytotoxicity of WT1469 (G2-8HQ) against DU-145: human prostate cancer studies line in the presence of Cu⁺² ions. Note the wide standard deviation at 1 and 0.5 μM indicating that in some % of the screens all the cancer cells were killed at these concentrations. The reason for the large standard deviations for W1469+Cu is discussed vide infra.

FIG. 4. Cytotoxicity of WT1469 (G2-8HQ) against BT-549: epithelial cells from ductal tumor in the presence of Cu⁺² ions. Note the wide standard deviation at 1 and 0.5 μM indicating that in some % of the screens all the cancer cells were killed at these concentrations. The reason for the large standard deviations for W1469+Cu is discussed vide infra.

FIG. 5. Cytotoxicity of WT1469 (G2-8HQ) against PANC-1: pancreatic cancer; in the presence of Cu⁺² ions. Note the wide standard deviation at 1 and 0.5 μM indicating that in some % of the screens all the cancer cells were killed at these concentrations. The reason for the large standard deviations for W1469+Cu is discussed vide infra.

FIG. 6. Cytotoxicity of WT1469 (G2-8HQ) against SU.86.86: a new adoptive pancreatic cell for immunotherapy studies in the presence of Cu⁺² ions. Note the wide standard deviation at 1 and 0.5 μM indicating that in some % of the screens all the cancer cells were killed at these concentrations. The reason for the large standard deviations for W1469+Cu is discussed vide infra.

FIG. 7. Cytotoxicity of WT1469 (G2-8HQ) against RL Cells (non-Hodgkin's lymphoma) in the presence of Cu⁺² ions. Note the wide standard deviation at 1 and 0.5 μM indicating that in some % of the screens all the cancer cells were killed at these concentrations. The reason for the large standard deviations for W1469+Cu is discussed vide infra.

FIG. 8. Proposed alternate novel intracellular conversion of G2-8HQ in the cytosolic matrix by low pH as predicted by our research. This hydrolysis reaction may be further enhanced by β-glucosidase to release 2-8HQ as an active Cu+2/Zn+2/Fe, etc. chelator. Note G2-8HQ under certain circumstances is also able to chelate metals without hydrolysis or enzymatic activation as shown elsewhere.

FIG. 9A W1469 copper complex in excipient, precipitation after 24 hours;

FIG. 9B Atomic structure determination by x-ray crystallography of dissolved and recrystallized (DMSO) precipitate.

FIG. 10. The complex of W1469p in aqueous solution, confirmed by crystallography. Where M=Mn(II), Fe(II), Co(II), Cu(II), Ni(II), and Zn (II) for W1469p

FIG. 11. The atomic structure of the ruby colored prismatic crystals prepared by dissolving the complex in FIG. 3 in DMSO and allowing the crystals to form over time. Structure determined under contract with John Hopkins University. Note: no water molecules are involved in the structure.

FIG. 12A. G2-8HQ (W1469) copper complex produced by the excipient/buffer mixture. Note the green color which is distinctly different than the yellow or red color of the 8HQ (W1469p) copper complex.

FIG. 12B Proposed copper complex of W1469 from aqueous synthesis based on determined copper complex structures of the 2,8HQ and 8HQ similarly produced.

FIG. 13. A cursory examination of the anti-proliferative activity of G2-8HQ administered as copper complex. It was noted by the individual performing the anti-proliferative study that significant material was lost during sterile filtration of G2-8HQ leading that person to state that he believed the biological activity of the complex was significantly better than observed.

FIG. 14. Biological Activity of 8-hydroxyquinoline (8HQ). Figure extracted from Gupta et al, 2021.

FIG. 15 (A) 8-Hydroxyquinoline (8HQ); (B) Copper complex of 8HQ with a metal:ligand ratio of 1:1; (C) Copper complex of 8HQ with a metal:ligand ratio of 1:2; (D) Nitroxoline; (E) Clioquinol; and (F) 8-Hydroxyquinoline-2-carboxylic acid (HCA).

FIG. 16 Uptake ratios tumor to blood after administration to BALB/c mice, implanted with murine Caco-2 colon adenocarcinoma tumor/blood rations.

FIG. 17. Synthetic Glucose derivatives of 8-hydroxyquinol (X=Z=H) (GuOHQ) and 5-chloro-7-iodo-8-hydroxyquinoline (X=I, Z=Cl) (GluCQ). Adapted from Oliver et al. 2012.

FIG. 18. IC50s values of GluCQ and Glu8HQ in three cell lines in the presence of copper(II). Human cell lines: A2780 (ovary, adenocarcinoma), A549 (lung, carcinoma) and MDA-MB-231 (breast, carcinoma). Excised from Oliveri, et al., 2012.

FIG. 19. Figure from Oliveri and Vecchio (2016) showing a selection of synthesized glucose and galactose derivatives.

FIG. 20. Trehalose derivatives of 8HQ at position 2.

FIG. 21. The keto-enol chemistry of 2, 8dihydroxyquinoline (2-8HQ).

FIG. 22. Predicted intracellular conversion of G2-8HQ by keto-enol chemistry at neutral to low pH and/or the β glucosidase hydrolysis reaction to release 2-8HQ as an active Cu+2/Zn+2 chelator.

FIG. 23: This is a photo of a patient in one of the clinical studies of the effectiveness of the treatment and compositions of the disclosure that was taken prior to treatment.

FIG. 24: This is a photo of a patient in one of the clinical studies of the effectiveness of the treatment and compositions of the disclosure that was taken following the course of a three-week treatment.

FIG. 25: This is a photo of a patient in another one of the clinical studies of the effectiveness of the treatment and compositions of the disclosure that was taken prior to treatment.

FIG. 26: This is a photo of a patient in another one of the clinical studies of the effectiveness of the treatment and compositions of the disclosure that was taken following one-week treatment.

FIGS. 27-30: These figures show NMR and Purity Spectra of 8-Hydroxy-2-quinolinyl beta-D-glucopyranosiduronic acid

### DETAILED DESCRIPTION OF EXEMPLARY EMBODIMENTS

As described herein, compositions and methods are provided for treating cancer and other conditions that comprise as the active ingredient 2,8-Di-hydroxyquinoline glucuronide and its derivatives, and more specifically the metabolic intermediate byproduct 8-Hydroxy-2-quinolinyl beta-D-glucopyranosiduronic acid (referred to herein as “G2-8HQ” and “W1469”). The compound 8-Hydroxy-2-quinolinyl beta-D-glucopyranosiduronic acid (which is also known as 2,8-Dihydroxyquinoline-beta-D-glucuronide) has the formula as shown below:

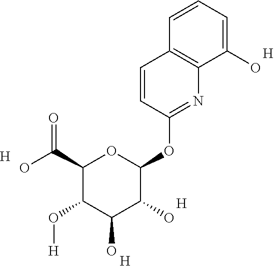

The invention also relates to the use of G2-8HQ, its stereoisomers and congeners, and other compounds with similar chemistry, such as those glucose or carbohydrate derivatives at positions 3-7 of the hydroxyquinoline ring. These compounds include those O-linked glucose or carbohydrate derivatives wherein the substitution is at the hydroxyquinoline positions 3-7 instead of the substitution at position 2 as in G2-8HQ. As described further herein, these compounds can be used individually or in combinations with omega fatty acids, and the therapeutic effectiveness of these compounds can be amplified in the presence of copper.

In addition, in accordance with the present invention, compositions and methods are provided for treating cancer and other conditions that comprise using as the active ingredient 2,8-quinolinediol (2-8HQ). The formula for 2,8-quinolinediol (also known as 2,8-dihydroxyquinoline) is as shown below:

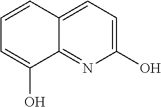 [SMILES: Oc1ccc2cccc(O)c2n1]

As described further herein, this compound or its natural enantiomeric tautomers, stereoisomers, or congeners can also be utilized in accordance with the present invention in compositions and methods used for treating cancers and a number of ailments as described further herein.

In accordance with the present disclosure, the details of exemplary non-limiting embodiments of the presently-disclosed subject matter are set forth herein. Modifications to embodiments described herein and other embodiments will be evident to those of ordinary skill in the art after a study of the information provided in this document. The information provided in this document, and particularly the specific details of the exemplary embodiments as described, is provided primarily for clearness of understanding and no unnecessary limitations are to be understood therefrom. While the terms used herein are believed to be well-understood by one of ordinary skill in the art, any definitions are set forth herein are provided to facilitate explanation of the presently-disclosed subject matter, but not to limit it in any way.

Unless defined otherwise, all technical and scientific terms used herein have the same meaning as commonly understood by one of ordinary skill in the art to which the presently-disclosed subject matter belongs. As one of ordinary skill in the art will understand, representative compositions, methods, and other aspects of the invention are now described, but other compositions, methods, and other aspects of the invention may be prepared that are similar or equivalent to those described herein and are considered practicing with the invention as now described.

Following long-standing patent law convention, the terms “a,” “an,” and “the” refer to “one or more” when used in this application, including the claims. Thus, for example, reference to “a subject” includes a plurality of such subjects, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

In addition, when used herein, if a range is indicated, any specific amount within that range is considered part of the invention. For example, wherein a range is given in mg/kg, this means that the range includes any individual amount within the range, for example if a range is provided as “10 μM to 100 μM”, all ranges within the parameter of that range are considered disclosed, and the range covers all individual amounts within that range, e.g., in 0.1 or 0.5 intervals. Accordingly, the inclusive range of “10 to 100” would include, for example, each 0.1 interval, such that the range would include a lower limit of about 10.1, 10.2, etc. in addition to about 10.0, and the upper range would include an upper limit of 99.9, 99.8, 99.7, etc., in addition to about 100.0. This would apply to all such ranges herein, in whatever units or measures are used.

Accordingly, the specific amount used may include any specific amount in the range, such as in 0.1 μM increments, so that amounts of 10.0, 10.1, 10.2, 10.3 and so forth up and including through 100.0 could be administered within the range of from about 10 to 100, and similarly for all ranges in accordance with the present invention.

As also referred to herein, the active compounds of the invention may be administered in the form of a pharmaceutical composition that may also include a pharmaceutically acceptable vehicle, carrier, or excipient. As used herein, the term “pharmaceutical vehicle, carrier or excipient” or “pharmaceutically acceptable vehicle, carrier or excipient” may refer to any of a wide variety of materials known for general usage in delivering a pharmaceutical agent or agents to a patient. Non-limiting examples of such materials include any solid forms that can be utilized in any suitable form of administration, such as oral administration, injection, intravenous (IV), suppository, topical, and spray/mist.

In certain exemplary embodiments, the pharmaceutical compositions of the invention may also include sterile aqueous or non-aqueous solutions, dispersions, suspensions or emulsions, as well as sterile powders for reconstitution into sterile injectable solutions or dispersions just prior to use.

As described further herein, the active hydroxyquinoline derivatives of the invention are used in methods of treating cancer and other conditions, such as viral infections, microbial infections, asthma, neurodegeneration, and inflammation. In such cases, the active ingredients of the invention will be administered to a subject in need thereof in an effective amount. In this respect, the term “effective amount” refers to an amount that is sufficient to achieve the desired treatment result or to have an effect on the targeted condition being treated. For example, an “effective amount” or a “therapeutically effective amount” refers to an amount that is sufficient to achieve the desired therapeutic result or to have an effect on undesired symptoms being targeted, but in such a manner as to achieve that objective but also reduce or eliminate the possibility of adverse side effects as would be recognized by those of ordinary skill in the art. The specific therapeutically effective dose level for use in the treatment of any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

Additionally, the terms “subject” or “subject in need thereof” or “patient” or “patient in need thereof” refer to a target of administration, which optionally displays symptoms related to a particular disease, pathological condition, disorder, or the like. The subject of the herein disclosed methods can be a human or animal patient, including mammals and other vertebrates in need of such treatment. The term subject or patient does not denote a particular age or sex and generally refers to a human or animal subject needing or receiving said treatment.

In one exemplary embodiment of the present disclosure, the present invention relates to a pharmaceutical composition comprising 8-Hydroxy-2-quinolinyl beta-D-glucopyranosiduronic acid (G2-8HQ) and a pharmaceutically acceptable vehicle, excipient, or carrier. As indicated herein, the term “pharmaceutical vehicle, carrier or excipient” or “pharmaceutically acceptable vehicle, carrier or excipient” may refer to any of a wide variety of materials known for general usage in delivering a pharmaceutical agent or agents to a patient. Non-limiting examples of such materials include any solid forms that can be utilized in any suitable form of administration, such as oral administration, injection, intravenous (IV), suppository, topical, and spray/mist forms.

As described further herein, the G2-8HQ of the invention may be administered alone or in the presence of a chelating metal, such as copper, of the ionic form of the chelating metal. As further described herein, although the presence of copper appears to enhance the anti-cancer ability and other therapeutic properties of G2-8HQ, there will normally be sufficient copper in the blood serum so that G2-8HQ will have suitable biological activity against cancer when administered to a subject in need thereof.

While it is possible to administer the G2-8HQ composition in the presence of copper, other suitable metal chelating ions can also be utilized, as would be recognized by one of ordinary skill in this art. For example, in addition to copper, the metal can be selected from the group consisting of copper, zinc, iron, cobalt, rhodium, and platinum. Further metals such as arsenic, antimony, gold, and vanadium could also be used in situations wherein the potential toxicity is reduced or eliminated.

The G2-8HQ pharmaceutical composition as described herein may also include a suitable phospholipid such as an omega fatty acid. For example, the omega fatty acid used may be an omega-3 fatty acid and/or an omega-6 fatty acid. In exemplary embodiments, the omega-3 fatty acid may be selected from the group consisting of alpha-linoleic acid (ALA), eicosapentaenoic acid (EPA), docosahexaenoic acid (DHA), and combinations thereof, and the omega-6 fatty acid may be selected from the group consisting of linoleic acid (LA), arachidonic acid (AA), docosapentaenoic acid (DPA), and combinations thereof. Even further, the G2-8HQ pharmaceutical composition can also include a hydroxyquinoline such as 8-hydroxyquinoline (8HQ).

The level of the G2-8HQ used in the present compositions will normally be that amount considered to be an effective amount against a particular condition of a patient in need of treatment thereof. As is described herein, the term “effective amount” refers to an amount that is sufficient to achieve the desired treatment result or to have an effect on the targeted condition being treated. For example, an “effective amount” or a “therapeutically effective amount” refers to an amount that is sufficient to achieve the desired therapeutic result or to have an effect on undesired symptoms being targeted, but in such a manner as to achieve that objective but also reduce or eliminate the possibility of adverse side effects as would be recognized by those of ordinary skill in the art.

The specific therapeutically effective dose level for use in the treatment of any particular patient will depend upon a variety of factors that can be determined by the doctor or other clinician and can include the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. In this regard, the G2-8HQ compositions of this disclosure may contain G2-8HQ at a concentration in the range of about 100 μM to about 500 mM. In other exemplary embodiments, this concentration may be in other suitable ranges, such as from about 300 μM to about 150 mM, from about 1 mM to about 50 mM, or from about 1 mM to about 25 mM.

In certain exemplary embodiments of the present disclosure, a method for treating cancer is provided which comprises administering an effective amount of a pharmaceutical composition comprising G2-8HQ and a pharmaceutically acceptable vehicle, carrier, or excipient to a subject in need thereof. As would be recognized by one of ordinary skill in the art, this administration can be in a number of suitable ways, including oral administration, injection, intravenous (IV), suppository, topical, and spray/mist, as would be determined by a doctor or other clinician based on the specific circumstances of the patient or subject being treated.

As indicated herein, the G2-8HQ pharmaceutical composition can also comprise or be administered with a chelating metal such as copper, zinc, iron, cobalt, rhodium, or platinum. In certain embodiments, the metal will be copper.

In another exemplary method of this disclosure, this pharmaceutical composition may also include or be administered with a phospholipid such as an omega fatty acid. The omega fatty acid may be an omega 3 fatty acid or an omega 6 fatty acid, and the omega fatty acids may be selected from the group consisting of alpha-linoleic acid (ALA), eicosapentaenoic acid (EPA), docosahexaenoic acid (DHA), linoleic acid (LA), arachidonic acid (AA), \docosapentaenoic acid (DPA), and combinations thereof.

As reflected above, G2-8HQ has shown properties that indicate it will have biological activity that can be used effectively to treat a variety of other conditions. In accordance with additional embodiments of the present disclosure, a method is provided for treating a condition selected from the group consisting of viral infection, a microbial infection, asthma, neurodegeneration, such as Alzheimer's disease, multiple sclerosis (MS), amyotrophic lateral sclerosis (ALS), or Parkinson's disease, and inflammation comprising administering an effective amount of a pharmaceutical composition comprising G2-8HQ and a pharmaceutically acceptable vehicle, carrier, or excipient to a subject in need thereof. As would be recognized by one of ordinary skill in the art, this administration can be in a number of suitable ways, including oral administration, injection, intravenous (IV), suppository, topical, and spray/mist, as would be determined by a doctor or other clinician based on the specific circumstances of the patient or subject being treated.

In certain embodiments, the pharmaceutical composition comprising G2-8HQ and a pharmaceutically acceptable vehicle, carrier, or excipient is administered along with a chelating metal. In certain embodiments, the chelating metal is selected from the group consisting of copper, zinc, iron, cobalt, rhodium, and platinum. In additional embodiments of the present invention, this pharmaceutical composition may also be administered along with a phospholipid such as an omega fatty acid. The omega fatty acid may be an omega 3 or 6 fatty acid such as alpha-linoleic acid (ALA), eicosapentaenoic acid (EPA), docosahexaenoic acid (DHA), linoleic acid (LA), arachidonic acid (AA), and/or docosapentaenoic acid (DPA), and/or combinations thereof.

In still further exemplary embodiments of the present disclosure, a pharmaceutical composition is provided that comprises 8-Hydroxy-2-quinolinyl beta-D-glucopyranosiduronic acid (G2-8HQ) and a compound selected from the group consisting of 8HQ, an omega-3 fatty acid, and an omega-6 fatty acid and a pharmaceutically acceptable vehicle, excipient, or carrier. In additional embodiments, a pharmaceutical composition is provided that comprises 8-Hydroxy-2-quinolinyl beta-D-glucopyranosiduronic acid and a chelating metal, and this chelating metal can be copper or any other suitable chelating metal, including but not limited to zinc, iron, cobalt, rhodium, and platinum.

As indicated herein, the pharmaceutical compositions comprise G2-8HQ as the active ingredient, and such compositions can be prepared using an effective amount of G2-8HQ that would be needed to treat a particular condition. In certain embodiments, the 8-Hydroxy-2-quinolinyl beta-D-glucopyranosiduronic acid (G2-8HQ) is at a concentration in the composition in the range of about 100 μM to about 500 mM. Alternatively, the amount of the G2-8HQ may also be in the range of from 300 μM to about 150 mM, from about 1 mM to about 50 mM, or from about 1 mM to about 25 mM. In the methods as disclosed herein, an effective amount of G2-8HQ is administered to a subject in need thereof, and such effective amounts may include concentrations in the ranges as described herein. As also described herein, the effective amounts of the active ingredients may be administered to a subject or patient in need thereof by any suitable method that would be determined by a doctor or clinician under the particular circumstances, and such administration may be selected from the group consisting of oral administration, injection, intravenous (IV), suppository, topical, and a spray/mist.

As also reflected herein, the pharmaceutical compositions comprising G2-8HQ and other compounds of the disclosure may be administered along with phospholipids such as omega fatty acids. As also indicated herein, the omega fatty acids can be selected from the group consisting of alpha-linoleic acid (ALA), eicosapentaenoic acid (EPA), docosahexaenoic acid (DHA), linoleic acid (LA), arachidonic acid (AA), docosapentaenoic acid (DPA), and combinations thereof.

In accordance with the present disclosure, a method of treating a condition selected from the group consisting of a viral infection, a microbial infection, asthma, neurodegeneration, and inflammation comprising administering an effective amount of the composition comprising 8-Hydroxy-2-quinolinyl beta-D-glucopyranosiduronic acid (G2-8HQ) and a chelating metal to a subject in need thereof. In certain embodiments, the chelating metal is selected from the group consisting of copper, zinc, iron, cobalt, rhodium, and platinum. In other embodiments, the composition to be administered in this method may also comprise an omega fatty acid or other phospholipid.

As indicated above, another hydroxyquinoline compound in accordance with this disclosure comprises 2,8-quinolinediol (2-8HQ) which may be utilized in the therapeutic methods as described herein. A pharmaceutical composition is also provided which comprises 2,8-quinolinediol (2-8HQ) and a pharmaceutically acceptable vehicle, excipient, or carrier. As indicated herein, the pharmaceutical composition utilizing 2-8HQ as the active ingredient may also comprise a chelating metal such as copper, or other common chelating metals including but not limited to zinc, iron, cobalt, rhodium, and platinum. Still further, this composition may also comprise an omega fatty acid, such as an omega-3 fatty acid and/or an omega-6 fatty acid. Such fatty acids may be selected from the group consisting of alpha-linoleic acid (ALA), eicosapentaenoic acid (EPA), docosahexaenoic acid (DHA), linoleic acid (LA), arachidonic acid (AA), docosapentaenoic acid (DPA), and combinations thereof. Still further, an additional hydroxyquinoline such as 8-hydroxyquinoline (8HQ) may also be added to this composition.

In another exemplary embodiment of the present disclosure, a pharmaceutical composition is provided that comprises 2,8-quinolinediol (2-8HQ) and another compound selected from the group consisting of 8HQ, an omega-3 fatty acid, and an omega-6 fatty acid, and a pharmaceutically acceptable vehicle, excipient, or carrier. Such a composition may further comprise a chelating metal such as copper, zinc, iron, cobalt, rhodium, and/or platinum. Such compositions can be utilized in the therapeutic methods as described herein.

Still further, a pharmaceutical composition is provided comprising 2,8-quinolinediol (2-8HQ) and a chelating metal. In certain embodiments, a method of treating cancer is provided that comprises administering an effective amount of this composition to a patient or subject in need thereof. Such administration may be carried out in a number of suitable ways, including oral administration, injection, intravenous (IV), suppository, topical, and/or by the use of a spray or mist, such as would be administered in an inhaler or other means of inhalation. As with the other active hydroxyquinoline compounds of the disclosure, the 2-8HQ compound may also be administered with a suitable chelating metal. The metals used along with the active compounds of the disclosure may be used in the form of ions, such as copper ions, zinc ions, iron ions, cobalt ions, rhodium ions, and/or platinum ions. Still further, these 2-8HQ pharmaceutical compositions may also include a phospholipid such as an omega fatty acid.

The G2-8HQ and 2-8HQ compounds of the present invention may be used in the therapeutic methods as described herein. For example, a method of treating cancer is provided which comprises administering an effective amount of G2-8HQ and/or 2-8HQ to a patent or subject in need of treatment thereof.

In additional exemplary embodiments, the G2-2HQ and 2-8HQ compounds of the present invention may be used in a method of treating a condition selected from the group consisting of a viral infection, a microbial infection, a parasitic infection, asthma, a neurodegenerative condition, and inflammation that comprises administering an effective amount of G2-@HQ and/or 2-8HQ to a patient or subject in need thereof. As indicated herein, such patients and subjects may be human or animal patients and subjects. The G2-8HQ and/or 2-8HQ compounds may be used in pharmaceutical compositions comprising the active ingredient along with a pharmaceutically acceptable carrier. In still other embodiments, the active G2-8HQ and 2-8HQ compounds may be administered with a chelating metal and/or a phospholipid such as an omega fatty acid.

In another exemplary embodiment of the present disclosure, a pharmaceutical composition complex is provided for use in treating a condition selected from the group consisting of cancer, a viral infection, a microbial infection, asthma, neurodegeneration, and inflammation comprising a glucuronic acid substituted 2,8-dihydroxy quinolone such as an 8-Hydroxy-2-quinolinyl beta-D-glucopyranosiduronic acid (G2-8HQ) and/or 2,8-quinolinediol (2-8HQ) in a pharmaceutically acceptable vehicle, carrier, or excipient, and such a composition may also include a chelating metal to form the complex.

In certain embodiments, a pharmaceutical composition complex is provided for use in treating a condition selected from the group consisting of cancer, a viral infection, a microbial infection, asthma, neurodegeneration, and inflammation comprising a glucuronic acid substituted 2,8-dihydroxy quinolone such as an 8-Hydroxy-2-quinolinyl beta-D-glucopyranosiduronic acid (G2-8HQ) and/or 2,8-quinolinediol (2-8HQ) in a pharmaceutically acceptable vehicle, carrier, or excipient, and such a composition may also include a chelating metal. The glucuronic acid substituted 2,8-dihydroxy quinolone may include any of a number of suitable compounds that contain substitutions at other positions on the hydroxyquinoline ring. Such glucuronic acid substituted 2,8-dihydroxy quinolone compounds may be selected from the group consisting of 8-Hydroxy-2-quinolinyl beta-D-glucopyranosiduronic acid, 8-Hydroxy-3-quinolinyl beta-D-glucopyranosiduronic acid, 8-Hydroxy-4-quinolinyl beta-D-glucopyranosiduronic acid, 8-Hydroxy-5-quinolinyl beta-D-glucopyranosiduronic acid, 8-Hydroxy-6-quinolinyl beta-D-glucopyranosiduronic acid, 8-Hydroxy-7-quinolinyl beta-D-glucopyranosiduronic acid, 2-beta-D-glucopyranosiduronic acid-5-chloro-7-iodo-8-hydroxyquinoline; 8-Hydroxy-2-quinolinyl beta-D-glucopyranoside, 8-Hydroxy-3-quinolinyl beta-D-glucopyranoside. 8-Hydroxy-4-quinolinyl beta-D-glucopyranoside, 8-Hydroxy-5-quinolinyl beta-D-glucopyranoside, 8-Hydroxy-6-quinolinyl beta-D-glucopyranoside, 8-Hydroxy-7-quinolinyl beta-D-glucopyranoside, and 2-beta-D-glucopyranoside-5-chloro-7-iodo-8-hydroxyquinoline. As with the other active hydroxyquinoline compounds described herein, these compounds may be administered in a complex wherein a chelating metal has been added.

In accordance with the present disclosure, another exemplary embodiment is provided that comprises a glucuronic acid derivative compound selected from the group consisting of 8-Hydroxy-2-quinolinyl beta-D-glucopyranosiduronic acid, 8-Hydroxy-3-quinolinyl beta-D-glucopyranosiduronic acid, 8-Hydroxy-4-quinolinyl beta-D-glucopyranosiduronic acid, 8-Hydroxy-5-quinolinyl beta-D-glucopyranosiduronic acid, 8-Hydroxy-6-quinolinyl beta-D-glucopyranosiduronic acid, 8-Hydroxy-7-quinolinyl beta-D-glucopyranosiduronic acid, 2-beta-D-glucopyranosiduronic acid-5-chloro-7-iodo-8-hydroxyquinoline; 8-Hydroxy-2-quinolinyl beta-D-glucopyranoside, 8-Hydroxy-3-quinolinyl beta-D-glucopyranoside. 8-Hydroxy-4-quinolinyl beta-D-glucopyranoside, 8-Hydroxy-5-quinolinyl beta-D-glucopyranoside, 8-Hydroxy-6-quinolinyl beta-D-glucopyranoside, 8-Hydroxy-7-quinolinyl beta-D-glucopyranoside, and 2-beta-D-glucopyranoside-5-chloro-7-iodo-8-hydroxyquinoline, and a pharmaceutically acceptable vehicle, excipient, or carrier. Such compositions may also comprise a chelating metal and/or other materials such as 8HQ and/or phospholipids such as an omega fatty acid.

In other exemplary embodiments of the present invention, a pharmaceutical composition is provided that comprises 8-Hydroxy-2-quinolinyl beta-D-glucopyranosiduronic acid (G2-8HQ) that is substituted with a different sugar at the 2-position, and a pharmaceutically acceptable vehicle, excipient, or carrier. In these embodiments, the sugar that is substituted can be any suitable sugar, including but not limited to galactose, glucose, fructose, sucrose, and trihalose. In another exemplary embodiment, a pharmaceutical composition is provided comprising 8-Hydroxy-2-quinolinyl beta-D-glucopyranosiduronic acid (G2-8HQ) modified so that an O-linked sugar is at a position other than at position 2, and a pharmaceutically acceptable vehicle, excipient, or carrier. In both of these embodiments, the compositions may also include a chelating metal, and/or a phospholipid such as an omega fatty acid.

In an additional exemplary embodiment, a pharmaceutical composition is provided that comprises a 2,8-dihydroxy quinolone glucuronic acid derivative and a pharmaceutically acceptable vehicle, carrier, or excipient. In certain embodiments, the glucuronic acid derivative is 8-Hydroxy-2-quinolinyl beta-D-glucopyranosiduronic acid, and in other embodiments, the glucuronic acid derivative is 8-Hydroxy-quinolinyl beta-D-glucopyranosiduronic acid. In all of these cases, these compositions can be used in effective amounts in methods of treating a condition selected from the group consisting of cancer, a viral infection, a microbial infection, asthma, neurodegeneration, and inflammation.

As reflected herein, the present compositions can be prepared through synthesis of the active ingredients as described herein. Alternatively, it is possible that G2-8HQ, 2-8HQ and the other hydroxyquinoline compounds of the invention can be obtained by isolation of these compounds from the serum of an ungulate and combining G2-8HQ with a pharmaceutically acceptable vehicle, carrier, or excipient. As indicated herein, these compounds are generally intermediate metabolic products found in the serum of ungulates such as goats, and these compounds may be isolated as described herein or by other methods known in the art of isolating compounds from blood and/or serum.

In accordance with the present disclosure, a method for use in treating cancer is provided comprising administering to a subject in need thereof an effective amount of a pharmaceutical composition comprising 8-Hydroxy-2-quinolinyl beta-D-glucopyranosiduronic acid (G2-8HQ) and a pharmaceutically acceptable vehicle, excipient, or carrier. In certain embodiments, the 8-Hydroxy-2-quinolinyl beta-D-glucopyranosiduronic acid is chelated with a metal. In additional embodiments, the 8-Hydroxy-2-quinolinyl beta-D-glucopyranosiduronic acid and the other suitable compounds of the invention may be administered along with serum plasma albumin, such as human serum albumin.

In other exemplary embodiments, a pharmaceutical composition for use in treating cancer comprising 8-Hydroxy-2-quinolinyl beta-D-glucopyranosiduronic acid (G2-8HQ) and a pharmaceutically acceptable vehicle, excipient, or carrier: As described further herein, the 8-Hydroxy-2-quinolinyl beta-D-glucopyranosiduronic acid may be chelated with a metal, and additionally, an omega fatty acid may also be added.

The active compounds and pharmaceutical compositions as described herein thus have suitable therapeutic properties so they can be utilized in a variety of therapeutic uses. For example, the use of an isolated 8-Hydroxy-2-quinolinyl beta-D-glucopyranosiduronic acid is provided for the treatment of cancer in a human or animal subject in need thereof. In such a use, the isolated 8-Hydroxy-2-quinolinyl beta-D-glucopyranosiduronic acid may be used in the form of a pharmaceutical composition wherein it is used along with a pharmaceutically acceptable vehicle, carrier, or excipient.

In additional uses in accordance with the present disclosure, there is provided a use of an isolated 8-Hydroxy-2-quinolinyl beta-D-glucopyranosiduronic acid for treatment of a condition selected from the group consisting of a viral infection, a microbial infection, a parasitic infection, asthma, neurodegeneration, and inflammation. In such a use, an effective amount of the isolated 8-Hydroxy-2-quinolinyl beta-D-glucopyranosiduronic acid is used with a pharmaceutically acceptable vehicle, carrier, or excipient, and in this composition, the isolated 8-Hydroxy-2-quinolinyl beta-D-glucopyranosiduronic acid may be used with a chelating metal.

Similarly, in additional exemplary embodiments of the present disclosure, there is provided the use of an isolated 2,8-quinolinediol (2-8HQ) is provided for the treatment of cancer in a human or animal subject in need thereof. In such a use, the isolated 2-8HQ may be used in the form of a pharmaceutical composition wherein it is used along with a pharmaceutically acceptable vehicle, carrier, or excipient.

In additional uses in accordance with the present disclosure, there is provided a use of an isolated 2-8HQ for treatment of a condition selected from the group consisting of a viral infection, a microbial infection, a parasitic infection, asthma, neurodegeneration, and inflammation. In such a use, an effective amount of the isolated 2-8HQ is used with a pharmaceutically acceptable vehicle, carrier, or excipient, and in this composition, the isolated 2-8HQ may be used along with a chelating metal.

In exemplary embodiments of formulations useable with the treatments and compositions in accordance with the present disclosure as follows:

### Formulations Summary

### (1) Topical Applications

All of the examples below would contain from about 0.5 to 4 mg/ml of W1469.

### Topical Example 1

Wherein the W1469 is dissolved in ethanol or aqueous buffered solutions thereof.

### Topical Example 2

More specifically, an aqueous ethanol (20% to 60%) buffered 0.05M to 0.2 M (HEPPES or PBS) from about pH 6.5 to 8.5, more specifically pH 7.4. If a gel is desired, this solution can be made with the addition of methylcellulose in amounts in order to achieve the desired gel strength according to common pharmaceutical compounding practices. In this case the ethanol at percentages 20% or greater can additionally serve as both a drug delivery and as an aseptic/preservative.

### Topical Example 3

Same as Example 1 with the addition of recombinant human serum albumin and/or sucrose/glucose at concentrations of 10 to 20 mg/ml as an excipient to aid in drug stabilization and skin penetration.

### Topical Example 4

Wherein the W1469 is dissolved in DMSO or aqueous buffered solutions thereof.

### Topical Example 5

Other pharmaceutically acceptable compounding bases pH adjusted, such as achievable with pluronic gels etc.

### (2) Injectable

Envisioned routes of administration include by conventional means and practice of those skilled in the art of establishing safety and efficacy and (formulation) and using drug delivery methodologies common to those practices (IV, IP, etc.). For example it is envisioned the drug may be incorporated in a sterile buffered low endotoxin PBS or other biologically compatible isotonic solution such as albumin.

### EXAMPLES

The presently-disclosed subject matter is further illustrated by the following specific but non-limiting examples. Some of the following examples are prophetic, notwithstanding the numerical values, results and/or data referred to and contained in the examples.

### Example 1

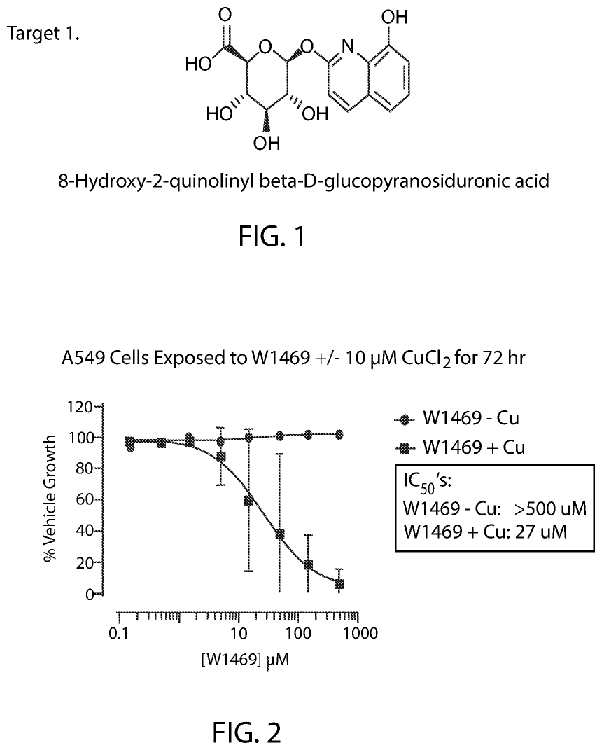

Although it has been known that several endogenous metabolic products have been associated with anti-cancer properties, an attempt was made to isolate, analyze, identify, and characterize some of these products from the serum plasma fraction of goats and other ungulates in order to examine the actual source of some of the properties shown. In addition to anti-cancer properties, some of the sera had exhibited extraordinary in vitro and in vivo activity against a large number of cancers and other targets, for example microbes such as mycobacteria tuberculosis and Cryptococcus, and viruses such as HIV and COVID 19. One of these products was the metabolite 8-hydroxyquinoline (8HQ) which has been associated with some anti-cancer and anti-proliferative activity. However, further studies resulted in the isolation and analysis of two other hydroxyquinoline compounds not previously associated with such activity. In particular, we isolated 8-Hydroxy-2-quinolinyl beta-D-glucopyranosiduronic acid (G2-8HQ) (FIG. 1) a metabolic product of 2,8-dihydroxyquinoline (2-8HQ), also known as 2,8 quinolinediol.

Initially the activity of these quinols against six cancer cell lines was determined where IC₅₀s ranged from 0.2 μM to >500 uM, and it was found that G2-8HQ exhibited advantages in anti-cancer activity and safety over 8HQ. In the case of G2-8HQ, the activity was more distinctly dependent upon the presence of copper (II) than 8HQ. For example, the IC₅₀ for G2-8HQ against the RL non-Hodgkin's lymphoma cell line in the presence of copper increased by more than 1000-fold (from >500 uM Cu⁺² to IC₅₀ 0.468 μM+Cu⁺²), and for 8HQ by 9-fold (IC₅₀ 0.06 μM+Cu⁺²), but these tests showed that G2-8HQ appeared to possess higher activity against cancer cell lines than 8HQ.

The experimental results thus showed that the presence of copper and the formation of the copper/metal complex appeared to be important in ensuring the anti-cancer activity of G2-8HQ, but the data also showed that G2-8HQ should have a greater safety margin (Therapeutic Index) than previously reported for any of the pro-drug glucose or glucuronide derivatives of 8HQ and its congeners, e.g., clioquinol, with comparable or superior anti-proliferative activity. Unlike previous pro-drug glucose or glucuronide derivatives which require enzymatic activation via β-glucosidase to release the active aglycone form, beta-glucosidase is not required by G2-8HQ. Additionally, G2-8HQ copper complex remains uncharacteristically, highly soluble in aqueous solutions. As evidenced by these results, G2-8HQ appears to be useful as an anti-cancer agent with greater solubility and a greater safety margin than what has been previously possible using 8HQ. Moreover, G2-8HQ contains a validated tumor targeting functionality which limits board systemic exposure and thus further improves both the safety and efficacy.

As an additional part of the data analyses to determine the biological basis for anticancer and antiviral activity present in the sera of selected goats, namely a selection of phospholipids comprising mainly of omega-3 and omega-6 polyunsaturated fatty acids with pre-characterized anti-cancer and immune modulatory properties including anti-inflammatory properties, were observed at concentrations up to >700× higher in positive animals (See Table 1 below). The omega-3 polyunsaturated fatty acids included alpha-linoleic acid (ALA), eicosapentaenoic acid (EPA) and docosahexaenoic acid (DHA), and the omega-6 polyunsaturated fatty acids included linoleic acid (LA), arachidonic acid (AA), and\docosapentaenoic acid (DPA).

TABLE 1
A selection of top differences in phospholipids
between two animals.

| | | % of | Of | Lipid | |
|---|---|---|---|---|---|
| LCP | P/N | Listed | Lipids | component | Property |
| 20:4 | 611 | 0.25 | 56.3% | Arachidonic | Immune |
| | | | | acid | modulatory |
| 20:4 | 743 | 0.313 | | Arachidonic | Immune |
| | | | | acid | modulatory |
| 22:6 | 297 | 0.125 | | Docosa- | Anti- |
| | | | | hexaenoic | cancer |
| | | | | acid | |
| 22:5* | 203 | 0.086 | | Docosa- | Anti- |
| | | | | pentaenoic | cancer |
| | | | | acid | |
| 18:3 | 180 | 0.076 | | Alpha- | |
| | | | | linoleic acid | |
| 18:2 | 115 | 0.048 | 8.7% | Conj. | Anti- |
| | | | | Linoleic acid | cancer |
| 22:6 | 22 | 0.009 | | Docosa- | Immune |
| | | | | hexaenoic | modulatory |
| | | | | acid | |
| PL | | | | | |
| 18:2 | 92 | 0.039 | | Conj. | Anti- |
| | | | | Linoleic acid | cancer |
| SPH | | | | | |
| d16 | 111 | 0.047 | | Sphingo- | |
| | | | | myelin | |

This information is of significance because it confirms previous studies such as with Clioquinol (CQ) which showed that its activity can be enhanced in the presence of docosahexaenoic acid (DHA). Further, the phospholipid forms of omega 3 and omega 6 fatty acids are known to have superior anti-cancer activity over their free fatty acid counterparts as shown in several prior studies (e.g., Fukunaga 2008; Hanaa 2014; Kafrawy 1998; and Mansara 2015). These synergies tend to support the theory that anticancer activity of metal complexes of hydroxyquinols is based on induced oxidative stress which is further enhanced by the presence of conjugated long chain fatty acids.

In the case of the quinolones, G2-8HQ has been identified, but not quantified or subject to further study relating to anticancer and other therapeutic properties. G2-8HQ is a metabolic intermediate product that has been identified in a variety of animals including poultry and rabbits in addition to ungulates such as horses, sheep, cattle, bison, deer, goats and swine. The principal locations for G2-8HQ appear to be in isolates from the blood, kidney and liver, and it is assumed that diet is the source for the primary compounds from which G2-8HQ is ultimately formed. For example, studies have identified 8HQ in the roots of some types of invasive knapweed plants, and this leads to the formation of 2-8HQ, a product of bacterial fermentation in the rumen in the case of ungulates. 2-8HQ is absorbed into the blood stream where it is further metabolized in the liver by UDP-glucuronosyltransferase (UDPGT) to yield G2-8HQ.

The presumed bacterial digestive origin of 2-8HQ is supported by its identification as a metabolite of 8HQ in the culture medium of at least one bacteria, Pseudomonas stutzeri. These compounds appear to be significantly elevated or induced in biologically active animal serum, and G2-8HQ has also been reported as one of the metabolic biomarkers for Peroxisome proliferator-activated receptor a (PPARα) expression and activation in mice (Zhen 2007).

The plasma concentrations of these compounds have not yet been determined quantitatively, but can be estimated in a range of approximately 100 μM to 5 mM based on the limitations of GC mass spec instrumentation utilized to identify them. We further estimate, based on the 23 fold higher concentration in a biologically active serum vs negative serum that these concentrations are in the range of 0.1 μM to about 1000 μM.

While the pharmaceutical properties of 8HQ have been extensively explored, other metabolite intermediate products such as G2-8HQ have not been similarly studied. As a result, the present inventors sought to determine the biological activity and physical properties of G2-8HQ in order to explore the glucuronic acid derivative as a unique and improved therapeutic, including its potential as a novel safer prodrug-like form. To date, there have been no studies relating to the anti-cancer, antimicrobial, anti-viral, anti-inflammatory and other therapeutic properties of G2-8HQ, which has been otherwise considered an obscure glucuronic acid intermediate metabolite.

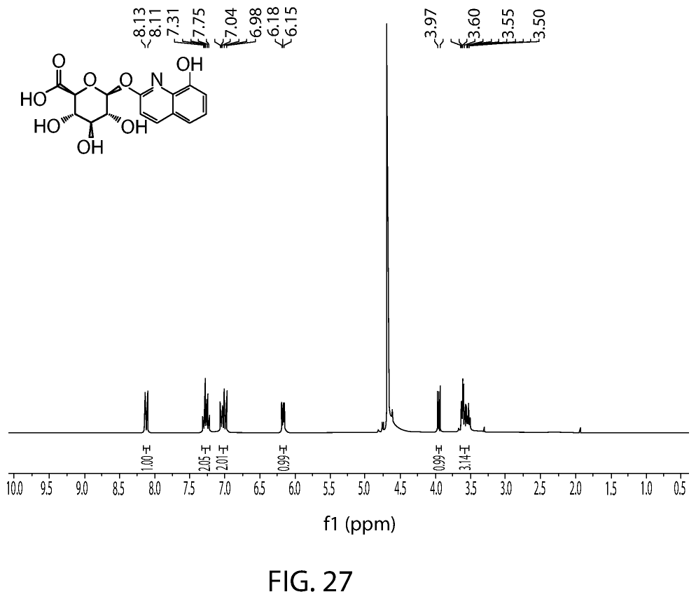

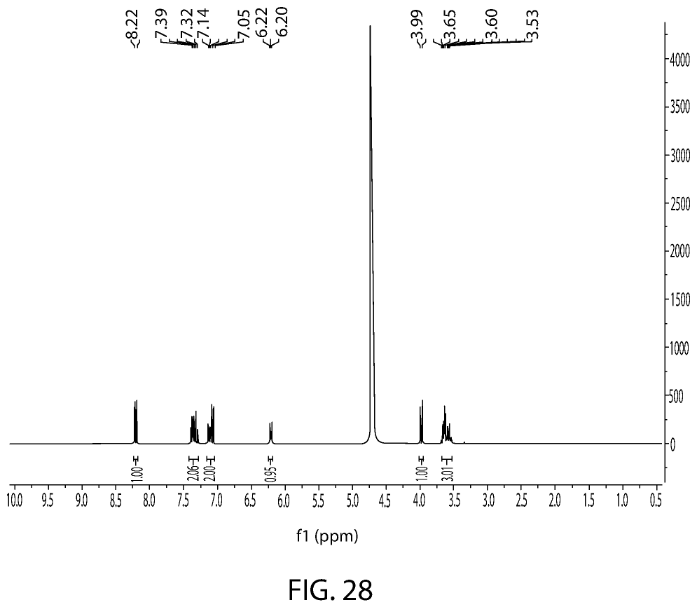

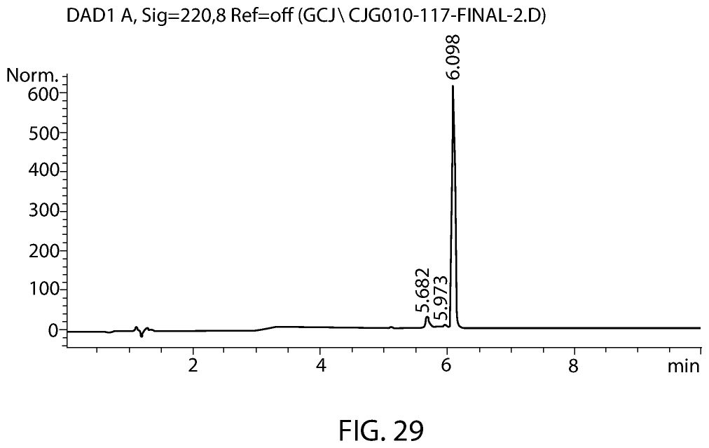

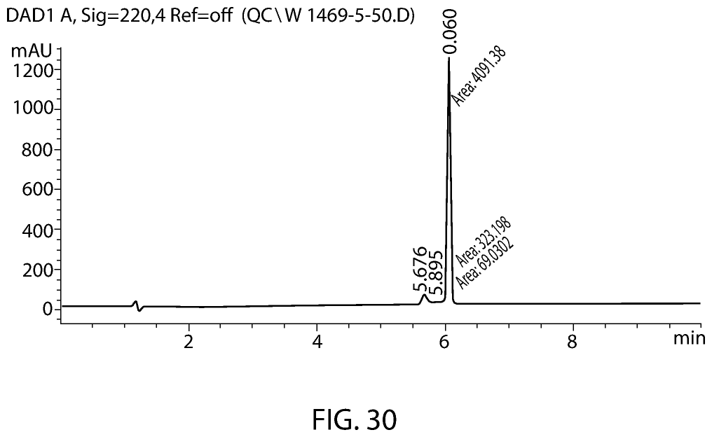

As a result, to study the potentially advantageous drug-like properties of G2-8HQ, we synthesized this compound and initiated the exploration of its properties as a novel chemotherapeutic agent. The G2-8HQ used in these studies were custom synthesized under contract to (1) Richman Chemical Co and (2) the University of Wisconsin, Milwaukee. The NMR and chromatographic purity of the synthetic G2-8HQ are shown in FIGS. 27-30. 8HQ and 2,8HQ also used in the studies were purchased commercially from Sigma Aldrich Chemical Company.

### Example 2

### Characterization of Anti-Cancer Properties of W1469 (G2-8HQ) and W1469p (8HQ)

Commercially available 8HQ and freshly synthesized G2-8HQ were prepared for in vitro anti-proliferative studies against the following cell lines with and without the presence of 10 μM Cu(+2): (1) A549: Breast cancer; (2) BT-549: epithelial cells from ductal tumor; (3) DU-145: human prostate cancer; (4) PANC-1: pancreatic cancer; (5) SU.86.86: a new adoptive pancreatic cell for immunotherapy studies; (6) RL: non-Hodgkin's lymphoma; (7) LnCAP, Prostate cancer cell line; (8) A431, a squamous cell carcinoma line; and (9) UACC-62 a malignant melanoma cell line. The details of the experimental protocol are provided below and these results are depicted in the FIGS. 2 through 10 and Example 9.

### Experimental Protocol for In Vitro Anti Proliferation Studies

With regard to the study protocol used, the objective was to conduct a study of the cytotoxicity of W1469 against several cancer cell lines. In the study protocol, preparations for the A549 (Lung) cell line screen are given as an example. The following materials were used: (1) FBS (2) RPMI-1640 medium supplemented with 10% FBS:’ and (3) A549 Cells. The four samples tested are referred to as Items 1-4 below and were placed at 4 °C upon arrival. These four samples were as follows:
Item 1: Tube A (W1469) (500 uM when FBS added)
Item 2: Tube B (W1469g) (1000 uM when FBS added)
Item 3: Tube C (W1469)
Item 4: Tube D (W1469g)

Items 1 and 2 are sterile ready for the addition of 1 ml of sterile FBS. Final concentrations after addition of 1 ml: 500 μM for Tube A and 1000 μM for Tube B. Items 3 and 4 are described further below. In the protocol, media containing 10% FBS and 20 μM CuCl2 are used in Tubes C and D only. A and B are serially diluted in media with 10% FBS and no added copper.

The protocol method proceeds as follows:

Seed 2, 96-well plates of A549 cells with 5,000 cells per well in a total volume of 50 μL per well. Leave overnight. See plate layout below for which wells to seed with 100 μL of media for media control. The following day, cells are exposed to compounds as described below.

For Items 1 & 2, to make the 500 μM and 1000 μM Stocks of samples 1 and 2 (W1469 and W1469g): Add 1 ml of sterile FBS to each Tube A and Tube B, swirl to mix. This results in Tube A at 500 μM stock and Tube B at 1000 μM.

To make appropriate 2× final dilutions of sample 1, Tube A (W1469), the following amounts are used:
500 μM: 10 mL as supplied
100 μM: 2 mL 500 μM Sample+8 mL media
30 μM: 3 mL 100 μM Sample+7 mL media
10 μM: 3 mL 30 μM Sample+6 mL media
3 μM: 3 mL 10 μM Sample+7 mL media
1 μM: 3 mL 3 μM Sample+6 mL media
0.3 μM: 3 mL 1 μM Sample+7 mL media
0.1 μM: 3 mL 0.3 μM Sample+6 mL media

To make appropriate 2× final dilutions of sample 2, Tube B (W1469g), the following amounts are used:
1000 μM: 10 mL as supplied
300 μM: 3 mL 1000 μM Sample+7 mL media
100 μM: 3 mL 300 μM Sample+6 mL media
30 μM: 3 mL 100 μM Sample+7 mL media
10 μM: 3 mL 30 μM Sample+6 mL media
3 μM: 3 mL 10 μM Sample+7 mL media
1 μM: 3 mL 3 μM Sample+6 mL media
0.3 μM: 3 mL 1 μM Sample+7 mL media

For Items 3 & 4, the following protocol was used to make 1000 uM Stocks of samples 3 and 4 (W1469 and W1469g):
1: Dissolve 3.37 mg W1469 and excipient (Tube C) in 1 ml RPMI medium without added FBS and copper and swirl briefly until completely dissolved. Add 9 ml of RPMI media containing 10% FBS and 20 uM copper. Swirl to mix. Sterile filter the 10 ml with provided syringes and filters. This is now the 1000 μM stock solution W1469 ready for screening
2: Dissolve 3.2 mg W1469g and excipient (Tube D) in 1 ml RPMI medium without added FBS and copper and swirl briefly until completely dissolved. Add 9 ml of RPMI media containing 10% FBS and 20 uM copper. Swirl to mix. Sterile filter the 10 ml with provided syringes and filters. This is now the 1000 μM stock solution W1469g ready for screening.

To make appropriate 2× final dilutions of samples 3 and 4, Tubes C and D (W1469 and W1469g respectively) were as follows:
1000 μM: 10 mL as prepared above
300 μM: 3 mL 1000 μM Sample+7 mL mix
100 μM: 3 mL 300 μM Sample+6 mL mix
30 μM: 3 mL 100 μM Sample+7 mL mix
10 μM: 3 mL 30 μM Sample+6 mL mix
3 μM: 3 mL 10 μM Sample+7 mL mix
1 μM: 3 mL 3 μM Sample+6 mL mix
0.3 μM: 3 mL 1 μM Sample+7 mL mix

To make 2 mM stock of CuCl2 in media, the following protocol was used:
2 mM CuCl2: Dissolve 34 mg CuCl2 in 100 mL media (Filter Sterilize) 20 μM CuCl2 media: 2 mL 2 mM CuCl2+198 mL media

Add 50 μL of above 2× stocks to appropriate wells to expose appropriate cells to the final dilutions of samples. Add 50 μL media to media and cell control wells and 50 μL media mix to vehicle control wells. Cells are exposed for 72 hr. Following a 72 hr exposure to Sample, perform a CTG assay on all remaining plates.

### CellTiter-Glo

At the end of the 72 hr exposure period, remove plates for CellTiter-Glo assay from 37 °C, 5% CO2 incubator and place on the bench at room temperature for 30 mins. Add 100 μL CellTiter-Glo reagent, mix for 2 mins, followed by a further 10 min incubation at room temperature. Record luminescence. Tables showing the resulting plate layouts are included below/

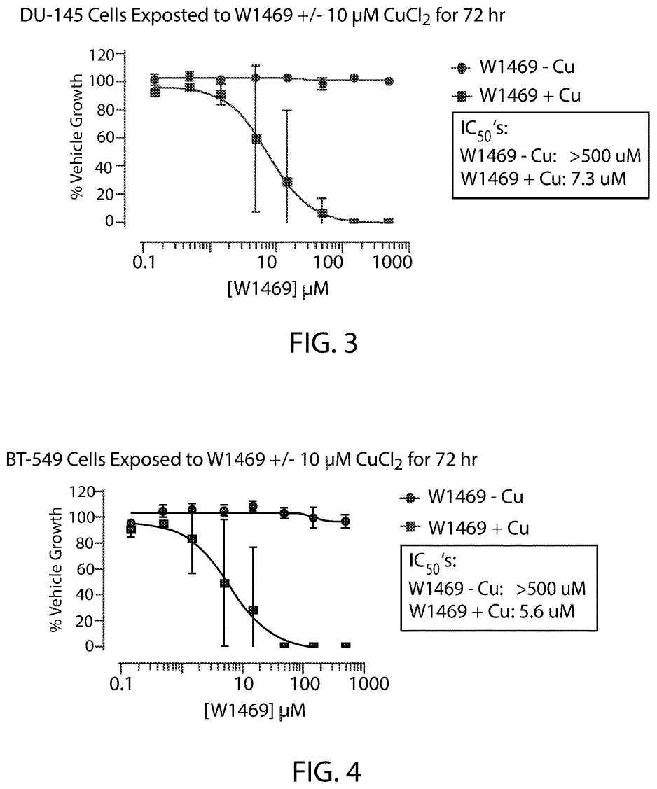

In these test results, FIG. 2 is a graphic representation of the cytotoxicity of WT1469 (G2-8HQ) against A549 breast cancer cell line in the presence of Cu⁺² ions. As indicated in FIG. 2, there is a wide standard deviation at 1 and 0.5 μM indicating lack of bioavailable copper in the cell media for complete copper chelation and further illustrating that in some percentage of the screens all the cancer cells were killed at these concentrations. Next, FIG. 3 reflects cytotoxicity of WT1469 (G2-8HQ) against the DU-145: human prostate cancer cell line, again in the presence of Cu⁺² ions. Again, there is a wide standard deviation at 1 and 0.5 μM indicating lack of bioavailable copper in the cell media for complete copper chelation and further illustrating that in some percentage of the screens all the cancer cells were killed at these concentrations.

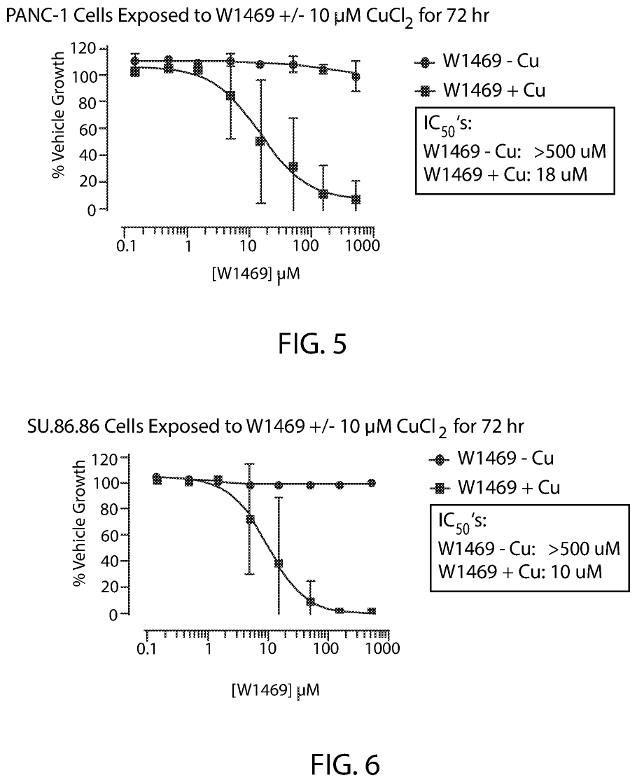

Next, FIG. 4 shows the cytotoxicity of WT1469 (G2-8HQ) against BT-549, namely epithelial cells from ductal tumor, in the presence of Cu⁺² ions. Again, there is a wide standard deviation at 1 and 0.5 μM indicating lack of bioavailable copper in the cell media for complete copper chelation and further illustrating that in some percentage of the screens all the cancer cells were killed at these concentrations. In FIG. 5, the tests were again related to determining the cytotoxicity of WT1469 (G2-8HQ) against PANC-1: pancreatic cancer, and this was again tested in the presence of Cu⁺² ions. The tests again indicated incomplete copper chelation by W1469 and that at 1 and 0.5 μM, at least some of the screens reflected that all the cancer cells were killed at these concentrations.

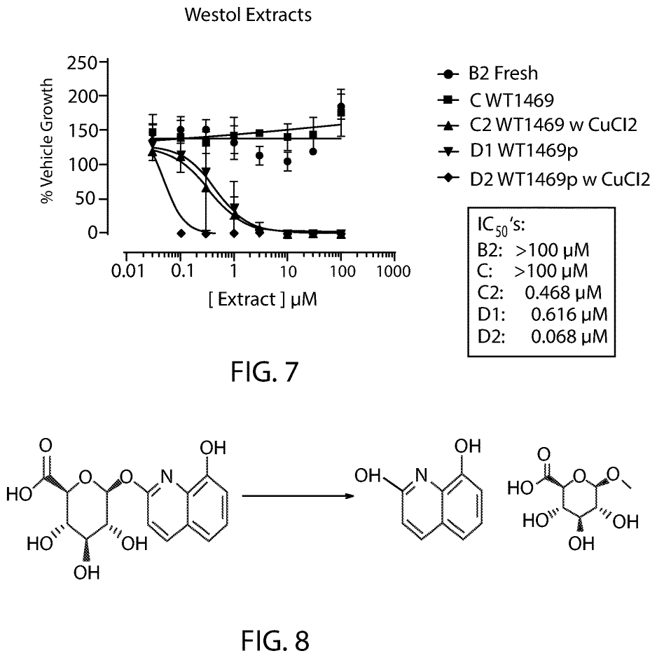

The other cancer cell lines tested gave similar results. As shown in FIG. 6, cytotoxicity was measured of WT1469 (G2-8HQ) against SU.86.86, a new adoptive pancreatic cell for immunotherapy studies, and these tests were conducted in the presence of Cu⁺² ions. FIG. 7 shows the results of cytotoxicity testing of WT1469 (G2-8HQ) against RL Cells (non-Hodgkin's lymphoma) in the presence of Cu⁺² ions. Again, in both cases, the testing showed that in a percentage of cases, all of the cancer cells were killed at concentrations between at 1 and 0.5 μM. Three additional cell lines were also studied and described more completely in Example 9 provided herein. These cell lines included cytotoxicity testing of WT1469 (G2-8HQ) against LnCAP a prostate cancer, A431 a squamous carcinoma cell line and UACC-62 a malignant melanoma cell line. All were complexed in situ with copper immediately prior to testing. The results from these additional cell lines and incorporated in Table 2 and Example 9.

Table 2 included below shows the IC₅₀ μM determinations for G2-8HQ and 8HQ against the following cancer cell lines, both with and without the presence of 10 μM Cu in the cell media: (1) A549: Breast cancer; (2) BT-549: epithelial cells from ductal tumor; (3) DU-145: human prostate cancer; (4) PANC-1: pancreatic cancer; (5) SU.86.86: a new adoptive pancreatic cell for immunotherapy studies; (6) RL: non-Hodgkin's lymphoma; (7) LnCAP: a prostate cancer cell line; (8) A431: a squamous cell carcinoma line; and (9) UACC-62: a malignant melanoma cell line. Additional supporting data is provided herein including NMR information.

TABLE 2
IC₅₀ μM determinations for G2-8HQ and 8HQ against nine cancer cell lines
IC₅₀ [uM]

| CMPD: | G2-8HQ | | | | 8HQ | | |
|---|---|---|---|---|---|---|---|
| Cell Line | −Cu | −Cu | +Cu | +Cu | −Cu | | +Cu |
| A549 | >500 μM | >500 μM | 27 μM | 63 μM | 12.7 μM | 29 μM | |
| BT-549 | >500 μM | >500 μM | 5.6 μM | 8.6 μM | 9.9 μM | 24.5 μM | |
| DU-145 | >500 μM | >500 μM | 7.3 μM | 8.7 μM | 11.6 μM | 11.6 μM | |
| PANC-1 | >500 μM | >500 μM | 18 μM | 39 μM | 9.3 μM | 11.2 μM | |
| Su.86.86 | >500 μM | >500 μM | 10 μM | 13 μM | >50 μM | 46 μM | |
| RL | >500 μM | | 0.47 μM | | 0.62 μM | | 0.07 μM |
| LnCAP | | | 109 μM | 154 μM | | | |
| A431 | | | 64 μM | 85 μM | | | |
| UACC-62 | | | 154 μM | 170 μM | | | |

A549: Lung Cancer
BT-549: Triple Negative Breast Cancer
DU-145: Prostate Cancer
PANC-1: Pancreatic Cancer
Su.86.86 Pancreatic Cancer
RL: Non Hodgkin's Lymphoma
LnCAP: Prostate Cancer
A431: Squamous Cell Carcinoma
UACC-62: Melanoma

It should be noted with the exception of the most recent experiments A431 and UACC-62, the calculations of IC50s in Table 2 are based on the W1469 MW of the apo form (337 MW) and not the actual active copper complex (771 MW). This means the IC50 values in Table 2 once recalculated will result in significantly lower (more active) IC50 values. These IC50 values have been updated in the table incorporated in Example 9. As can be seen in Table 2, G2-8HQ in the presence of copper shows significant cytotoxicity against the cancer cell lines. With the addition of either 10 or 20 μM Cu⁺², the cytotoxicity towards various cancer cell lines markedly increases ranging from >>than 1000-fold increase with the RL—non-Hodgkin's lymphoma, >>90 fold for BT549, >>68-fold for DU-145 prostate cancer, to >>8-18 fold improvement with A549.

### Example 3

### Studies of Solubility and Chemical Properties of W1469 (G2-8HQ) and W1469p (8HQ)

Under certain conditions, G2-8HQ, although highly soluble, in water or PBS (at neutral pH), will not readily complex Cu⁺², which sometimes leads to reduced levels of toxicity and anti-proliferative activity. It is believed that intra or inter molecular interactions of the glucuronic acid may in some cases sterically hinder access to the 8-hydroxy. As a result, a suitable excipient will normally be needed to ensure that steric hindrance is disrupted as described further herein.

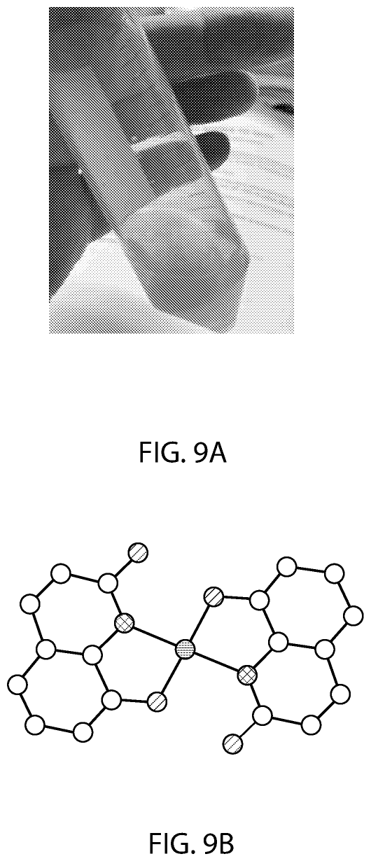

In general, it has been observed that since G2-8HQ is less stable in aqueous solutions at pH values lower than 8 or over time in organic solvents, it is often necessary to ensure proper conditions of pH in order to maximize G2-8HQ stability. For example a pH of 5 has been shown to degrade the compound almost immediately, while dissolving in DMSO (without copper) yields over time the precipitated degradation product of the keto form of 2-8HQ (we determined by x-ray crystallography). Similarly, the G2-8HQ copper complex at pH values less than 8, results in rapid precipitated insoluble 2-8HQ copper complex by ejecting the glucuronic acid adduct to yield a copper complex of 2-8HQ (determined by x-ray crystallography under contract at John Hopkins University and shown in FIG. 9 and described herein).

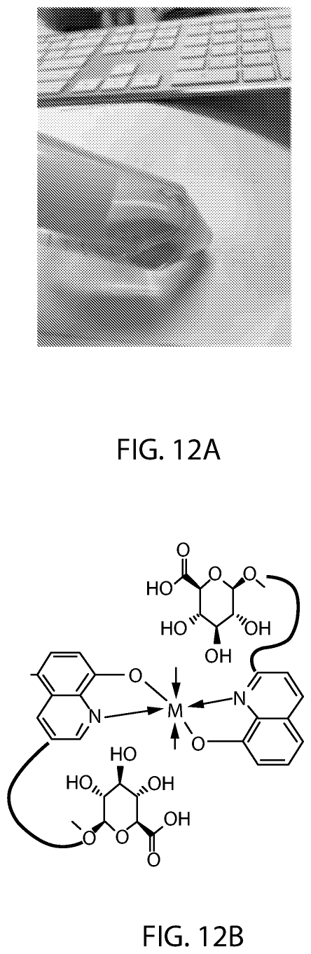

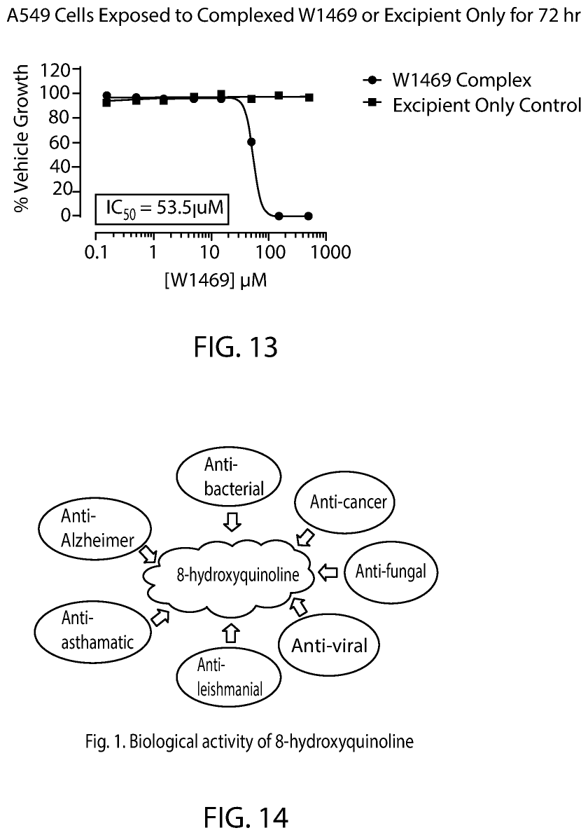

In light of the foregoing, a green stable biologically active form of G2-8HQ was prepared as a copper complex using an excipient mixture inclusive of aqueous buffers at pH 8-8.2. The structure for this complex is shown in FIG. 12.

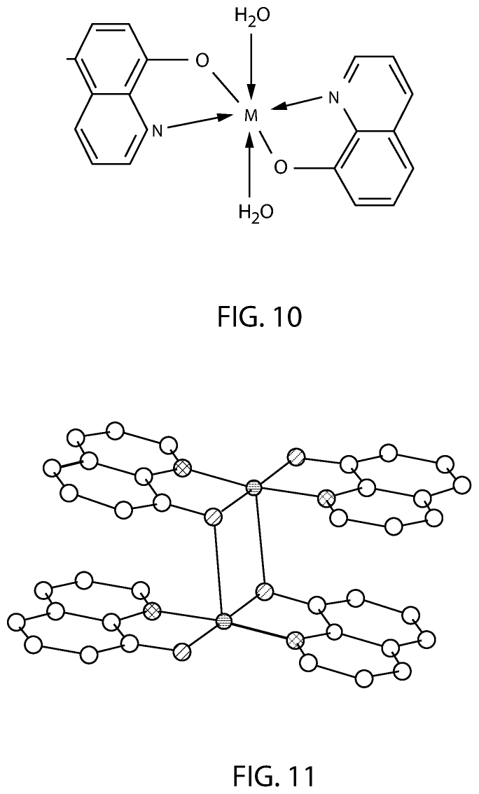

On the other hand, 8HQ is sparingly soluble in water, but will generally be soluble in DMSO. The 8HQ compound can rapidly form a copper complex in aqueous solutions to yield an aqueous insoluble bright yellow precipitate. This precipitate, when dissolved in DMSO, yields dark red prismatic crystals under slow evaporation. The atomic structure of this complex was determined by x-ray crystallography under contract at John Hopkins and is shown in FIG. 11. This structure may have important implications of the in vivo and in vitro activity of 8HQ, and its divalent copper structure may have superior ROS generation over other 8HQ analogs, whereas the crystallization from more aqueous alcohol solutions yields the classic structure which includes two water molecules (see FIG. 10).

As shown herein in the drawing figures, as referred to above, FIG. 9A shows the G2-8HQ (W1469) copper complex in an excipient, with precipitation after 24 hours. FIG. 9B is an atomic structure determination by x-ray crystallography of dissolved and recrystallized (DMSO) G2-8HQ (W1469) precipitate. In FIG. 10, the complex of W1469p (8HQ) in aqueous solution is shown as confirmed by crystallography. In the graphs, M=Mn(II), Fe(II), Co(II), Cu(II), Ni(II), and Zn (II) for W1469p. In FIG. 11, the atomic structure of the ruby colored prismatic crystals prepared by dissolving the complex in FIG. 3 as described above in DMSO and then allowing the crystals to form over time. The structure was determined under contract with John Hopkins University, and no water molecules are involved in the crystalline structure.

These tests have shown the necessary chemical properties and potential usefulness of G2-8HQ in anti-cancer and other therapeutic methods as described herein.

### Example 4

### Alternate Novel Intracellular Conversion of G2-8HQ

As shown herein in FIG. 8, an alternate novel intracellular conversion of G2-8HQ is possible using the following reaction:

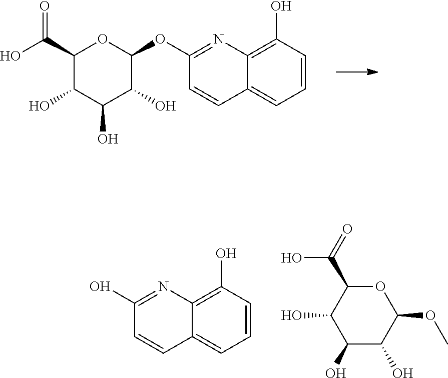 [SMILES: O=C(O)[C@H]1O[C@@H](Oc2ccc3cccc(O)c3n2)[C@H](O)[C@@H](O)[C@@H]1O ; Oc1ccc2cccc(O)c2n1 ; CO[C@@H]1O[C@H](C(=O)O)[C@@H](O)[C@H](O)[C@H]1O]

This proposed intracellular conversion of G2-8HQ in the cytosolic matrix is carried out in low pH conditions as predicted by our research. This hydrolysis reaction may be further enhanced by β-glucosidase to release 2-8HQ as an active Cu+2/Zn+2/Fe, etc. chelator. In addition, under certain circumstances, G2-8HQ is also able to chelate metals without hydrolysis or enzymatic activation as shown herein.

### Example 5

### Studies of the Pre-Formed Copper Complex of G2-8HQ

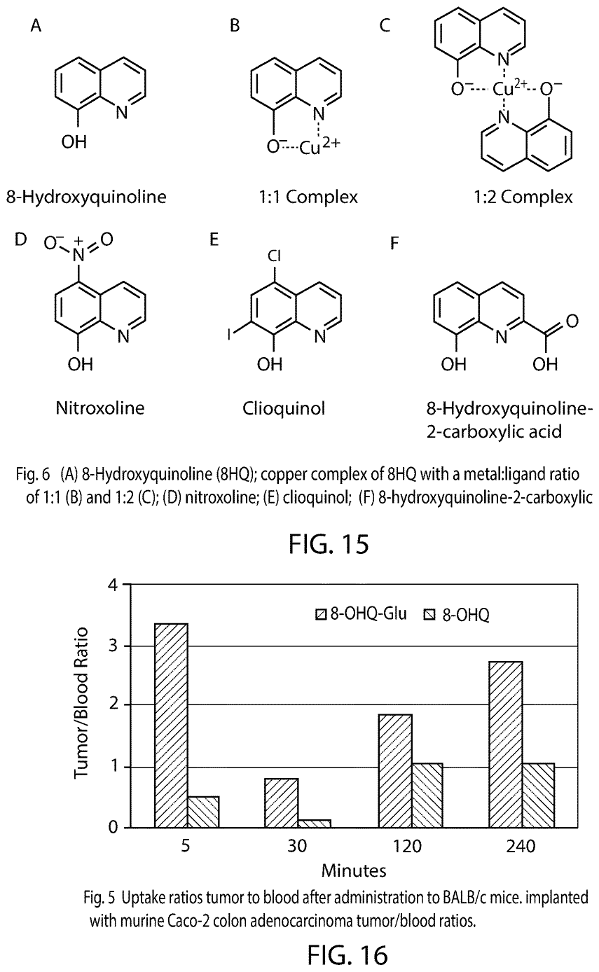

Additional testing was conducted with regard to the pre-complexed copper complex as shown in FIG. 12B herein based on the new determination that G2-8HQ could bind copper without enzymatic activation, and these tests were conducted against A549 to determine the nature of the unusually large standard deviations. The results are shown in FIG. 13 and compared with FIG. 2 reveal that the previous large standard deviations are now essentially eliminated and suggesting a variability of structure conformational chemistry interfering with copper coordination in prior experiments. Two separate A549 assays produced virtually identical results of IC50 53.5 and 54 μM consistent with the accuracy implied by the low standard deviations. However, it was noted during the experiment that significant material was lost during sterile filtration which affected the desired solution concentrations, strongly suggesting that the IC50s were actually much better.

In accordance with these tests, FIG. 12A is provided which shows the G2-8HQ (W1469) copper complex produced by the excipient/buffer mixture as described herein. This has a green color which is distinctly different than the yellow or red color of the 8HQ (W1469p) copper complex as also depicted herein. FIG. 12B shows the proposed copper complex of W1469 from aqueous synthesis based on determined copper complex structures of the 2,8HQ and 8HQ similarly produced. In FIG. 13, a cursory examination was made of the anti-proliferative activity of G2-8HQ administered as a copper complex. It was noted in the anti-proliferative study that significant material was lost during sterile filtration of G2-8HQ evidencing that the biological activity of the complex was even significantly better than observed, and perhaps significantly so. In short, rather than disrupting copper homeostasis, these results support the theory that these metal complexes act as ROS centers inducing apoptosis through oxidative stress. Once again, the tests evidenced the anti-proliferative properties of G2-8HQ.

### Example 6

### G2-8HQ and its Alternatives, and Summary and Advantages Over Prior Art

As shown in the specification and examples described herein, G2-8HQ provides a new anti-cancer, anti-viral, anti-bacterial, anti-parasitic, anti-neurodegenerative, anti-fungal agent with enhanced and highly desirable efficacy, safety, and clinical administrative characteristics. This provides for the first time a direct ring modified O-linked glucose or glucuronic acid (or any sugar) at the 2 position of 8HQ has been studied for use as a therapeutic platform. As shown herein, G2-8HQ has unique desirable solubility and pro-drug like properties with the further expectation of enhanced safety profile and in the case of cancer applications, improved tumor partitioning. As shown herein, G2-8HQ has a powerful anticancer activity against all cell lines tested and further shows superior biological activity when enhanced using copper chelation. As shown herein, the testing as described herein was primarily done using copper chelation, but it would be understood that different metals could be substituted to achieve various desired activities against different cell lines and organisms, such metals include but are not limited to Cu, Zn, Fe, Co, Rh, Pt, etc. In addition, when used at suitable levels that will not cause patient toxicity, other heavy metals such as arsenic, antimony, gold, vanadium in addition to platinum and iron have traditionally been used among cancer chemotherapy agents and thus could also be used in G2-8HQ complexes as set forth in this disclosure.

In light of the testing described herein, there are several conclusions that can be drawn when comparing G2-8HQ with the prior art studies of others relating to glucose or glucuronic acid derivatives of 8HQ quinols. For example, based on the work described by Monson 1991 and Yesilagac 2011, there was some indication of possible activity using 8HQ and its related compounds, but there were no studies and thus no knowledge of the possible use of G2-8HQ with regard to anti-proliferation activity.

The unique properties of G2-8HQ as discovered by the present inventors and studied herein revealed that G2-8HQ and additional unique glucose or carbohydrate derivatives at the 8HQ positions 2-7 have the desired solubility and targeting as well as an absence of the requirement of metabolic activation (i.e., they are not pro-drugs). The use of the compounds individually or in combination with phospholipid compounds are thus described herein, and these uses include a variety of therapeutic methods to treat cancers and other ailments. It would further be understood that in accordance with the present invention, substitution of other sugars at the 2 position (such as galactose, glucose, fructose, sucrose, trihalose, etc.) and any congeners of a sugar-2-8HQ form thereof are also contemplated within the invention. These other related compounds are included in Table 3 below.

TABLE 3
Examples of related chemical structures taking advantage of the
disclosed 2-8HQ chemistry.

| | |
|---|---|
| 8-Hydroxy-2-quinolinyl beta-D- | 8-Hydroxy-2-quinolinyl |
| glucopyranosiduronic acid | beta-D-glucopyranoside |
| 8-Hydroxy-3-quinolinyl beta-D- | 8-Hydroxy-3-quinolinyl |
| glucopyranosiduronic acid | beta-D-glucopyranoside |
| 8-Hydroxy-4-quinolinyl beta-D- | 8-Hydroxy-4-quinolinyl |
| glucopyranosiduronic acid | beta-D-glucopyranoside |
| 8-Hydroxy-5-quinolinyl beta-D- | 8-Hydroxy-5-quinolinyl |
| glucopyranosiduronic acid | beta-D-glucopyranoside |
| 8-Hydroxy-6-quinolinyl beta-D- | 8-Hydroxy-6-quinolinyl |
| glucopyranosiduronic acid | beta-D-glucopyranoside |
| 8-Hydroxy-7-quinolinyl beta-D- | 8-Hydroxy-7-quinolinyl |
| glucopyranosiduronic acid | beta-D-glucopyranoside |
| 2-beta-D-glucopyranosiduronic acid | 2-beta-D-glucopyranoside- |
| -5-chloro-7-iodo-8-hydroxyquinoline | 5-chloro-7-iodo-8- |
| | hydroxyquinoline |

### Example 7

### Summaries of the Prior Art and Unique Advantages of G2-8HQ Over Prior Art

Previously, much of the state-of-the-art of 8HQ-based pharmaceutical development has been recently and extensively reviewed in Gupta 2021 and FIG. 14 included herein illustrates the broad range of potentially beneficial biological activity. Some of the compounds referenced therein are shown in FIG. 15. The function of 8HQ and its derivatives has largely been attributed to their inherent chromophore heavy metal chelating properties, most notably Cu, Fe and Zn. In some cases the function is associated with the direct metal interference, such as in the inhibition of metalloprotease and for others the alteration in transition metal homeostasis or the creation of ROS (free radical, oxidative stress strongly implicated). However, due to difficulties in developing some of these compounds for use in anti-cancer methods, the use of 8HQs has still been greatly restricted.

Still other possible applications of the 8HQs have been examined due to the possibility of other activities outside of anti-cancer activity. For example, it has been raised that compounds such as CQ also show activity against viruses such as COVID 19, but again the use of CQ has been stopped due to problems of toxicity. Such activity may be attributable to blocking the viral attachment to ACE receptors required for viral spike protein mediated infection. Copper chelation does not appear to be required for the anti-viral function.

In Table 4 below, taken from Monson 1991, the tissue content of 8HQ (ng/g tissue) after tumor acidification and subsequent Glu8HQ administration is shown.

TABLE 4
Tissue content of 8HQ (ng/g tissue) after tumor acidification and
subsequent Glu8HQ administration (Monson 1991)
Table 1. Tissue content of 8-OHQ (ng/g tissue) after tumor acidification and
subsequent 8-OHQ-glucuronide administration*

| Tissue | 10 min | 30 min | 45 min | 60 min | 90 min |
|---|---|---|---|---|---|
| RIF tumor^{†} | 40 ± 3 (3) | 94 ± 28 (4) | 116 ± 11 (3) | 104 ± 25 (3) | 57 ± 9 (3) |
| Kidney | (4)^{‡} | 94 ± 43 (3) | 39 ± 6 (3) | (3)^{‡} | (4)^{‡} |
| Liver | 23 ± 14 (4) | 26 ± 12 (3) | 19 ± 8 (3) | 5 ± 5 (3) | (4)^{‡} |
| Spleen | (3)^{‡} | (3)^{‡} | — | 20 ± 16 (4) | (3)^{‡} |
| Colon | 57 ± 37 (5) | 50 ± 32 (5) | 57 ± 33 (3) | 7 ± 6 (4) | (4)^{‡} |
| Duodenum | (4)^{‡} | (4)^{‡} | (3)^{‡} | 37 ± 19 (3) | (3)^{‡} |
| Muscle | (4)^{‡} | 12 ± 10(3) | 16 ± (30) | 27 ± 12 (3) | 13 ± 11 (3) |
| Brain | 3)^{‡} | (3)^{‡} | (3)^{‡} | (3)^{‡} | (3)^{‡} |
| Gallbladder | — | 3947 ± 991 (4) | 192 ± 91 (3) | — | — |
| Colon contents | — | 242 ± 44 (3) | 243 ± 38 (4) | 119 ± 93 (3) | 182 ± 21 (2) |

*The table does not show other data obtained for all of the above tissues at 2.0, 2.5, 3, 4, 6, 8, and 24 hr after glucuronide injection.
These data points, with n = 3 animals, also failed to show significant tissue levels of 8-OHQ.
^{†}The tumor was implanted on the dorsal aspect of the hind foot.
^{‡}Below the limits of detection (<5 ng/g tissue). Numbers in parentheses represent values for n, the number of independent repeat experiments. Hyphens indicate that no measurements were made.

In Monson 1991, there was an exploration of the potential of anti-tumor targeting utilizing the glucuronide derivations of pharmaceuticals using G8HQ as a model system. It seems this was their primary purpose rather than advocating the prodrug form as a cancer therapeutic. This work documented several important aspects of glucuronide G8HQ concerning tumor targeting, and the biodistributions of the active aglycone, 8HQ, was examined in tissues of mice at various time points. However, in their initial animal experiments they found no significant tumor or general tissue partitioning, and this clouded future research in this area. Another finding was that prior acidification of tumors by the induction of hyperglycemia was required. After acidification, tumor uptake was rapid and significantly increased showing significant tumor targeting and G8HQ activation, and this is summarized in Table 4 above. Some indication of anti-tumor activity was shown, but these were brief experiments over the course of 24 hours after which point the study and animals were terminated.

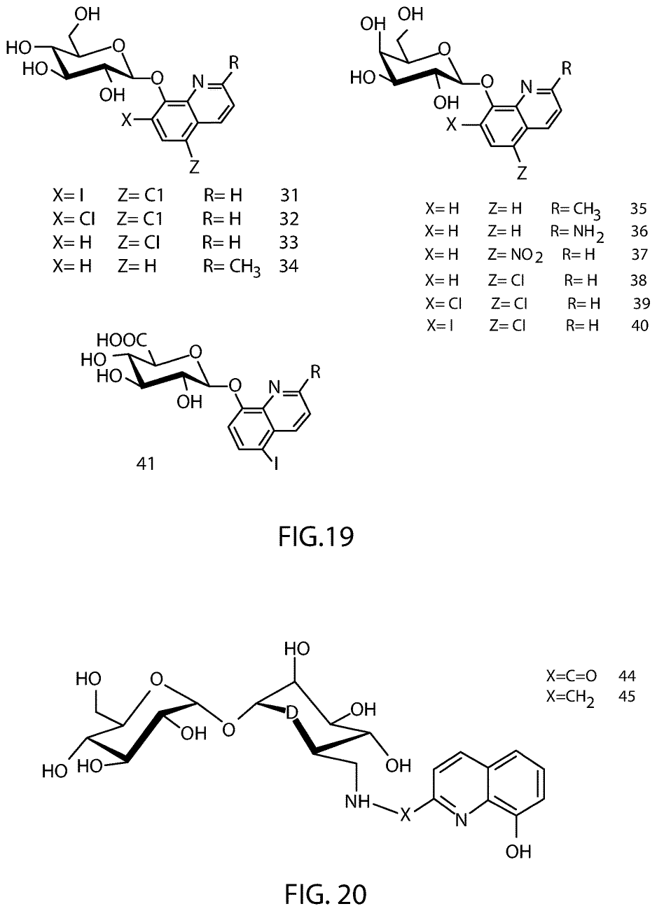

Many years later, Yesilagac 2011, explored (4-iodo, 8-hydroxy beta-D-glucopyranosiduronic acid by incorporating radioactive iodine to examine bio distribution and demonstrate the potential for both radiation diagnostics and radiation treatment. Their bio distribution was similar to Monson 1991, but distinct since an iodinated derivative (compound 41 in FIG. 19) would not be expected to have the same bio distribution and pharmacokinetic properties as the 8HQ. Both Yesilagac 2011 and Monson 1991 found evidence for enteropathic recirculation which (as shown in FIG. 16 herein) may prolong the half-life, with Yesilagac determining that excretion of the iodinated drug is mainly urinary, and Monson 1991 evidencing that the 8GHQ/8HQ drug is largely eliminated at 3 hours post injection.

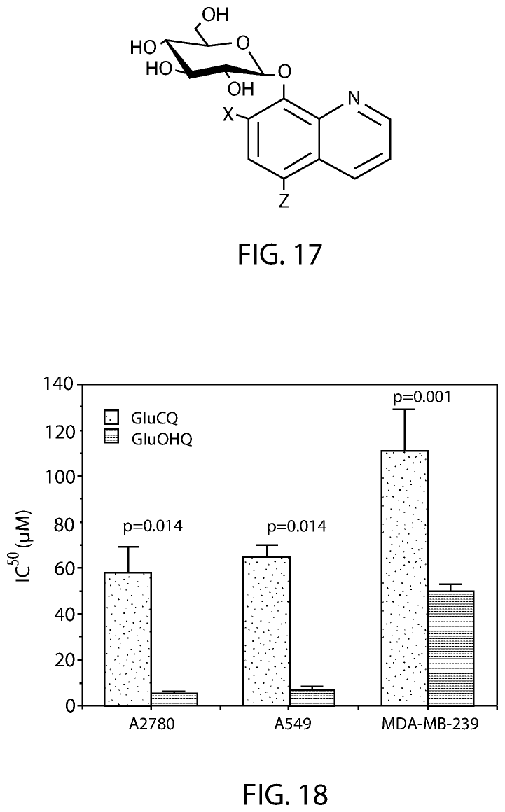

The present inventors obtained a commercial sample of G8HQ and tested it in vitro under conditions with copper added and found a possible reading of >500 uM IC 50, indicating no anti-proliferative activity, but such examination was cursory and not conclusive. Of further relevance, Oliveri 2012 designed an O-linked glucose adducts to the 8-hydroxy position of both 8HQ and CQ (FIG. 17). The resulting pro-drug form as expected abrogated the copper binding and eliminated the anti-cancer activity. The authors reasoned that the glucose adduct function as an inactive prodrug to be converted to the active 8HQ form by the hydrolysis of the glycosidic bond by beta-glucosidases in the cytoplasm of cancer cells. The authors reasoned that the glucose adduct would improve solubility, and the glucose receptors over-populated on the surface of cancer cells. The anaerobic metabolism and high dependence on glucose that characterizes most cancers is a phenomenon known as the “Warburg Effect”.

Still other testing also had mixed results. Oliveri 2012 also examined their glucose derivatives performance against three Human cell lines: A2780 (ovary, adenocarcinoma), A549 (lung, carcinoma) and MDA-MB-231 (breast, carcinoma) as shown in FIG. 18 and demonstrated there was a significant increase in activity through the addition of Cu⁺². Additionally, they tested glucose adducts to the 8-hydroxyl position (prodrug) and indicated that these are activated intracellularly in the cytoplasm by Beta-glucosidases, releasing the metal binding and potent anti-proliferative activity of 8HQ (see FIG. 17 and compounds 31-34 in FIG. 19). In later studies, O-linked chlorinated derivative compounds 32 and 33 also showed enhanced cytotoxicity in the presence of copper. Although not explicitly stated by Oliveri, the elevated copper concentrations found in the blood of many cancer patients has been determined by others to be approximately 20 μM, which was assumed to be the basis for the selection of this concentration in their studies. The Oliveri results for synthetic Glucose derivatives of 8-hydroxyquinol (X=Z=H) (Glu8HQ) and 5-chloro-7-iodo-8-hydroxyquinoline (X=I, Z=Cl) (GluCQ) are illustrated in FIG. 18 and demonstrate that certain compounds such as a glucose-modified 8HQ (Glu8HQ) have greater activity than the CQ analog (GluCQ). FIG. 19 also illustrates a number of 8HQ derivatives synthesized and studied for their antiproliferative properties.

In other studies, the galactose modifications at the 8-hydroxy position (compounds 35-40) were not able to be cleaved by the beta glucosidase and resulted in a loss of cytotoxicity, as reflected in Oliveri 2016. Moreover, in the same review, it was noted that trehalose derivatives at position 2 incorporated by either an amide or amine moiety (FIG. 20), as well as linked polysaccharides at a variety of frame positions also show little or no antiproliferative activity and the inclusion of copper ions did not affect their activity. The authors proposed that these derivatives were not recognized by the GLUTs or were too polar to pass through the cellular membrane.

In all of these cases there was some uncertainty concerning the nature of the 8HQ activity and drawbacks to their use in therapeutic methods that are overcome using the compounds of the present invention as described herein.

For example, another unique distinction of G2-8HQ over the prior art involves the adaptation of the 2,8HQ as the active copper chelating quinolone frame, vs. the simple 8HQ. The compound 2,8HQ, unlike 8HQ, has the disadvantage of poor aqueous solubility, which is counterintuitive based on the additional hydroxyl at position 2.

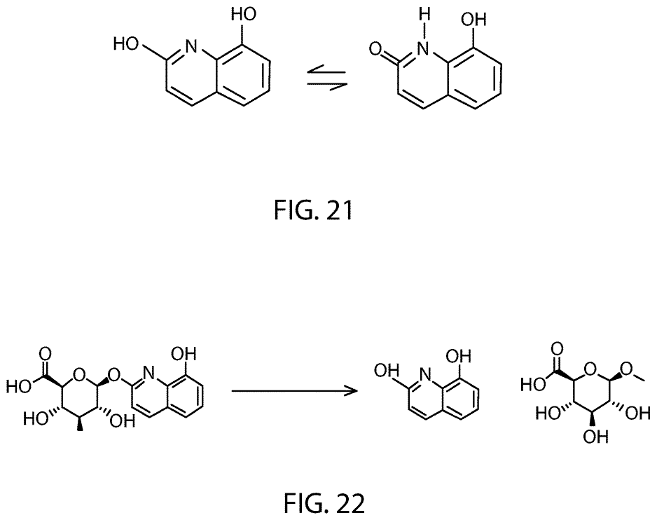

However, the present inventors have discovered that the 2-hydroxy has unique properties among all 8-hydroxy quinolone frame derivatives in its inherent keto-enol chemistry, as shown in FIG. 21.

The keto-enol chemistry of 2,8-dihydroxyquinoline (2-8HQ) (FIG. 21) is as follows:

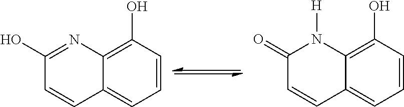 [SMILES: Oc1ccc2cccc(O)c2n1 ; O=c1ccc2cccc(O)c2[nH]1]

As a result, unlike the prior art involving 8-hydroxyquinolines and glucose or glucuronide derivatives, where the primary focus was on prodrug forms which release the aglycone drug form enzymatically by beta-glucosidase, the compounds of the present invention including G2-8HQ are active without beta-glucosidase Further, although not required in the case of G2-8HQ the reactive chemistry/instability of the glucuronide at position 2 yields a second pathway at acidic cytosolic pH, to release or “kick-off” the sugar moiety, which in principal should further enhance the antiproliferative effect is shown in FIG. 22. In the predicted intracellular conversion of G2-8HQ by keto-enol chemistry at neutral to low pH and/or the β glucosidase hydrolysis reaction to release 2-8HQ as an additional active Cu+2/Zn+2 chelator form is shown as follows:

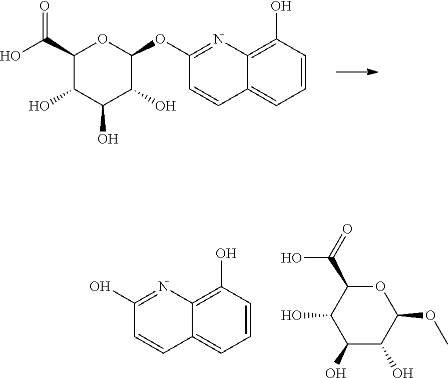 [SMILES: O=C(O)[C@H]1O[C@@H](Oc2ccc3cccc(O)c3n2)[C@H](O)[C@@H](O)[C@@H]1O ; Oc1ccc2cccc(O)c2n1 ; CO[C@@H]1O[C@H](C(=O)O)[C@@H](O)[C@H](O)[C@H]1O]

In another distinction over prior art as shown for example in Monson and Oliveri, the present inventors have shown herein that G2-8HQ can under appropriate conditions, chelate copper without enzymatic or keto-enol hydrolysis. Under these conditions the high standard deviations observed in our prior in vitro screening as described herein disappears leaving no doubt that the G2-8HQ has antiproliferative activity unknown in the prior art, and that this activity is greatly enhanced by copper or other chelating metals. This shows that unlike any of the prior art such as described by Monson and Oliveri, G2-8HQ can be administered with the glucuronic acid/glucose-based targeting intact. Furthermore, unlike hydrolyzed copper complexes of the activated drugs using Glu8HQ and G8HQ, G2-8HQ remains highly aqueous soluble.

In summary, (1) the prior art to date has not uncovered or examined the special O-linked chemistry of the 8-hydroxyquinoline frame disclosed here; (2) there is also an absence in the prior art of any antiproliferative or other potential therapeutic studies of the 2,8HQ parent compound or G2-8HQ; and (3) it would be understood by those skilled in the art that the application of the keto-enol chemistry in accordance with this invention can be applied as discussed herein to a variety of sugars, as well as congeners of the 8-hydroxy frame, and such compounds can be used effectively in anti-cancer, anti-proliferative, and other therapeutic applications.

### Example 8

### Additional Advantages of G2-8HQ and its Antiproliferative Activity Over the Prior Art

As indicated above, there were previous findings relating to the indication of potential anti-cancer activity for compounds such as the glucose 8 of CQ and 8HQ. The anticancer activities of these compounds were first examined against A2780, A549, and MDA-MB-231 with and without copper and with and without glucose modification O-linked at the 8-hydroxy position. They reported unmodified 8HQ was about 9 to 27× higher in activity than the comparable tests with CQ. These trends were carried over in the glucose modifications, but with comparatively 10-fold lower activity, which further indicated the uncertainty with the results. Combined with the toxicity issue relating to the use of CQ, this uncertainty restricted efforts to move ahead with the use of such technology.

In other studies, in the presence of 20 μM Cu⁺², the compound Glu8HQ had 10× higher activity compared with Glu8CQ. Without copper, anti-proliferative activity was still observed but at much lower levels—for Glu8CQ the improvement with the addition of copper was only about 2-fold, whereas for Glu8HQ it was roughly 10-20×. Once again, even with regard to the effect of the presence of copper ions, it was not clear what the precise effect was with regard to the use of these compounds in the presence of copper, although varying levels of improved activity had been shown.

With regard to the use of the G2-8HQ of the present disclosure, a comparison with the prior art (a glucuronide vs glucose), it was observed that for tests against six of the nine carcinoma cell lines tested without the inclusion of copper as described herein, IC50s were all greater than 500 μM indicating a lack of drug cytotoxicity in the Apo form (see Table 2 above). This reflects a potentially dramatic improvement over the Apo studies of 8HQ analogs of the type conducted by Oliveri. Using the G2-8HQ compounds of the present discloser, with the addition of either 10 or 20 μM Cu⁺², the cytotoxicity towards various cancer cell lines markedly increases ranging from a greater than 1000-fold increase with the RL—non-Hodgkin's lymphoma, a greater than 90-fold increase for BT549, a greater than 68-fold increase for DU-145 prostate cancer, and a greater than 8-18 fold improvement with A459. These results are markedly superior to those reported by Oliveri 2011 even at higher copper concentrations (20 μM Cu⁺²).

Accordingly, the present inventors have shown that since anticancer and anti-microbial activities of 8HQ pharmacophores require copper to be effective, pre-complexation with copper would be highly desirable administrative form, and the ability to locate and use more suitable forms in this regard, such as G2-8HQ, is a huge advantage in the potential uses of these compounds for therapeutic purposes. For example, it has also been found that metal complexes cannot form if the glucose or glucuronide derivative is in place at the 8OH, and thus the prior art compounds synthesized and evaluated by Oliveri & Manson make pre-complexation impossible. Moreover, the copper complexes of 8-hydroxy quinolone and its congeners are highly insoluble in aqueous solutions which also makes them less desirable in this form. Further, as indicated above, compounds like CQ that previously were halted as anti-cancer drugs because of toxicity and undesirable side effects, and thus it is important to develop new compounds in this area which can improve the safety of the administration and reduce or eliminate serious side effects.

The problems associated with the prior art are greatly avoided with the G2-8HQ of the present invention which, in the glucuronide tumor targeting form, can in aqueous solution yield highly soluble and active compounds, such as shown in FIG. 10. Once again, it has been shown that G2-8HQ has: 1) superior solubility with or without complexed copper, and 2) superior and unique safety potential and biological activity that overcomes the previous issues with 8HQ as reflected in many previous studies.

In this regard, addition in vitro experiments (without acidification) with 8GHQ that showed no activity, and in other studies, the metastasis of tumors in triple negative was greatly reduced by copper reduction and did not result in any reduction in tumor size. Such results again show that G2-8HQ as an un-complexed drug could serve two roles, namely (1) reduction of bioavailable copper and (2) anticancer treatment. The present data on the elevated copper levels in the blood serum of cancer patients seems to indicate that they are around 23 uM, and at such a level, the serum copper is more than sufficient to activate G2-8HQ without pre-complexation. In other words, in accordance with the present invention, therapeutic methods are provided wherein the G2-8HQ is administered in a form that does not complex it with copper ions in advance, and thus even when administered alone, G2-8HQ can be utilized effectively against cancer and other conditions as described in detail herein. A summary of additional comparative advantages of G2-8HQ over the art is shown below in Table 5.

TABLE 5
Comparative advantages and differences of G2-8HQ over prior art

| | Active | | | | | |
|---|---|---|---|---|---|---|
| | w/o Cu | Complexes | Solubility of Cu | Require Beta | Targets | Parent |
| Compound | (cancer) | w/copper | Complex/aqueous | glucosidase | tumor | Drug |
| G2-8HQ | No | Yes | excellent | No | Yes | 2,8HQ |
| GA8HQ | No | No | None, (8HQ poor) | Yes | Yes | 8HQ |
| | | | | Requires tumor | | |
| | | | | acidification in | | |
| | | | | vivo | | |
| G8HQ | No | No | None | Yes | Yes | 8HQ |

As reflected herein, several strategies for treatment and modes of delivery are possible in light of the showing herein regarding chemistry and safety profile of G2-8HQ. As also reflected above, this represents the first time the pharmaceutical potential of metabolic beta-D-glucopyranosiduronic acid derivatives of 2,8-dihydroxyquinolines have been examined, and the first time that the advantages in using the present compounds for therapeutic purposes such as in treating cancer have been documented.

In this regard, additional tests were conducted showing the superior beneficial results of treatment in accordance with the present disclosure.

### Example 9

### Additional Testing to Set Up Case Studies Using the Compounds of the Disclosure

### Overview

In additional testing, we were able to continue demonstrating the expected broad anticancer activity of the compounds of the disclosure in three additional in vitro cell lines, including skin cancers, in order to test the compounds in specific patient clinical studies.

### Additional In Vitro Screens

The compounds were tested in conjunction with the following conditions, and the graphs showing the results in each case are included below:

### Additional Tables of Results:

From the original list tables referred to above of in vitro assay results, additional test results showing the effectiveness of the claimed compounds are now included. In the table below, nine cell lines were tested, and all show various levels of anticancer activity.

### Adjustment of Tables of Results:

Since all the previous screens compiled in Table 2 were conducted where the MW of W1469 used was 337 and this did not reflect the actual MW of the active species which requires complexation with copper, a bi-dentate coupling. We have shown above that at least with regard to anticancer activity, the apo form is not active in vitro, and thus the MW of the copper complex is 776 reflective of the molecular formula, where the 5^{th} and 6^{th} ligands to the copper are believed to be hydroxides based on crystallographic evidence. Thus, the actual uM activities can be adjusted to more closely approximate true values by multiplying each value by 337/776=0.43. As a result of these adjustments, a revised Table with adjusted values based on the MW of the copper complex as calculated above is presented below:

IC₅₀ [μM]

| CMPD: | W1469 | | |
|---|---|---|---|
| Cell Line | −Cu | +Cu | +Cu |
| | 1 | 2 | 3 |
| A549 | >500 μM | 11.6 μM | 27.1 μM |
| BT-549 | >500 μM | 2.4 μM | 3.7 μM |
| DU-145 | >500 μM | 3.1 μM | 3.7 μM |
| PANC-1 | >500 μM | 7.7 μM | 16.8 μM |
| Su.86.86 | >500 μM | 4.3 μM | 5.6 μM |
| RL | >500 μM | 0.20 μM | |
| LnCAP | | 46.9 μM* | 66.2 μM* |
| A431 | | 63.2 μM* | 83.4 μM* |
| UACC- | | 152.2 μM* | 167.3 μM* |
| 62 | | | |

Column 1: IC50 values for cell lines tested with Apo form of W1469; Column 2: In complexed with Cu; Column 3: duplicate test of Column 2 where applicable.
(*) Denotes differences with or without excipient added and early suboptimal attempts at complexing the copper in situ just prior to the cell line screening. It is expected that these IC50 values will improve significantly and be more commensurate with the earlier screens, once the procedure is optimized to minimize degradation during the complexation.

The nine cell lines tested as shown above were as again as follows:
A549: Lung Cancer
BT-549: Triple Negative Breast Cancer
DU-145: Prostate Cancer
PANC-1: Pancreatic Cancer
Su.86.86 Pancreatic Cancer
RL: Non Hodgkin's Lymphoma
LnCAP: Prostate Cancer
A431: Squamous Cell Carcinoma
UACC-62: Melanoma

### Example 10

### Case Studies Using the Compounds of the Disclosure

### Exploratory Clinical Studies of Basal Cell and Squamous Cell Cancer

Skin cancer is the most common form of cancer in the United States and more people are diagnosed with skin cancer each year in the U.S. than all other cancers combined.
Research estimates that non-melanoma skin cancer (NMSC), including basal cell carcinoma (BCC) and squamous cell carcinoma (SCC), affects more than 3 million Americans a year. It is estimated that approximately 9,500 people in the U.S. are diagnosed with skin cancer every day. It is further estimated that the overall incidence of BCC has increased by 145% between 1976-1984 and 2000-2010, and the overall incidence of SCC increased 263% over that same period. Actinic keratosis is the most common pre-cancer; it affects more than 58 million Americans.
When such conditions are diagnosed, the current standard of care includes surgical excision (the most common method prescribed), cryogenics, topical treatments and radiation treatments. Of these, the most common prescribed topical treatments are shown below:

| | |
|---|---|
| Photodynamic Therapy: | Aminolaevulinic acid (Levulan)* |
| Side Effects: | Burning, Itching or Tingling, Rash or redness |
| | Light sensitivity (bright lights/sunshine) |
| Topical Chemotherapy: | Fluorouracil 4 to 12 wks |
| Side Effects: | Burning or itching, rash or redness, sore or |
| | tender skin, serious allergic reaction |
| | to drug (rare) |
| Immune response modifier: | Zyclara, Alara (Imiquimod) up to 8 wks |
| Side Effects: | Redness, bleeding or flaking skin, swelling, |
| | blistering, sting or pain. Fatigue or tiredness, |
| | headache or backpain, Diarrhea |

However, the majority of treatments can leave the patient scarred or disfigured and thus there is a growing need for patient friendly, safer, more effective topical treatments for skin cancer. Topical treatments using photodynamic therapy or immunomodulatory drugs exhibit serious systemic side effects. All current topical methodologies have serious limitations in effectiveness. Thus, the more common preferred treatment is surgical removal which can result in the scarring or disfiguring discussed above. As a result, there is a need for a more effective treatment protocol against such skin cancers, and the case studies below were conducted based on the compounds as described herein.

### Single Patient Clinical Study Design:

Indication: Basil Cell Carcinoma
Rationale: Basil Cell Carcinoma is the most common form of skin cancer with 3.6 Million cases diagnosed each year and rising. It is estimated that 80% of all newly diagnosed skin cancers are BCC. Although rarely fatal, it can be painful and disfiguring and, in some cases, become dangerously invasive.
   The most common forms of treatment include surgery, radiation treatment, immunotherapy, systemic and topical chemotherapy, and other variations including laser therapy. The currently approved topical treatments for BCC are usually only recommended for more superficial tumors and more extensive monitoring is required because they are generally not effective in destroying cancer cell deeper below the surface. A more complete description of treatment options and their relative effectiveness has been described by the American Cancer Society.
   There is a need for a safer, more effective topical treatments of BCC which can potentially be used by individual patients outside the clinical setting. And if necessary, on a continuing ongoing basis.

### Study Approach:

The examined treatment approach studied here falls in the category of a topical chemotherapeutic. W1469 is a novel small molecule anti-cancer drug candidate which contains a tumor targeting functional group and has been shown effective against a variety of cancers in vitro. (most recently including squamous cell carcinoma)

### Drug Vehicle:

Topical gel containing 0.5 mg/ml W1469 in proprietary aqueous alcohol gel, applied initially once daily using a suitable topical applicator. As the studies progressed and no toxicity was observed, higher dosing concentrations (4 mg/ml) and drug vehicles of ethanol or DMSO alone were further studied.

### Treatment Protocol:

In general, the Treatment/Patient protocol used for the patient testing was as follows:
1. Photograph the application site prior to starting the protocol. Note any changes in lesion size, color, etc. of treatment area.
2. Remove a loaded syringe from the freezer, dry ice or refrigerator and allow to gradually warm to room temperature.
3. Photograph the syringe each time prior to application. Note the color of the gel contained in the syringe.
4. Lightly clean the skin surface with alcohol wipe.
5. With clean hands, unscrew the syringe cap (Note: these syringes do not contain needles), place the syringe tip on the application site and slowly dispense a small amount of gel sufficient to cover the treatment area (assuming the diameter of a penny or nickel). not rub this in. Let the liquid from the gel absorb into the site without disturbing. Apply once a day at the same time.
6. Re-cap the syringe and store in the refrigerator until completely used.
7. Repeat 1-6 each daily during application period.
   It is noted in this protocol that photographing the syringe used with each application period accomplishes two things. It shows if there are any issues with patient compliance, and it allows the clinician to follow any color changes in the W1469 which may indicate loss of activity.

### Study Objectives:

1. Toleration/toxicity
2. Application methodology
3. Any visual indications of efficacy
4. Patient feedback
   Study Duration: Varied case by case. Generally, two weeks to two months

### Clinical Results:

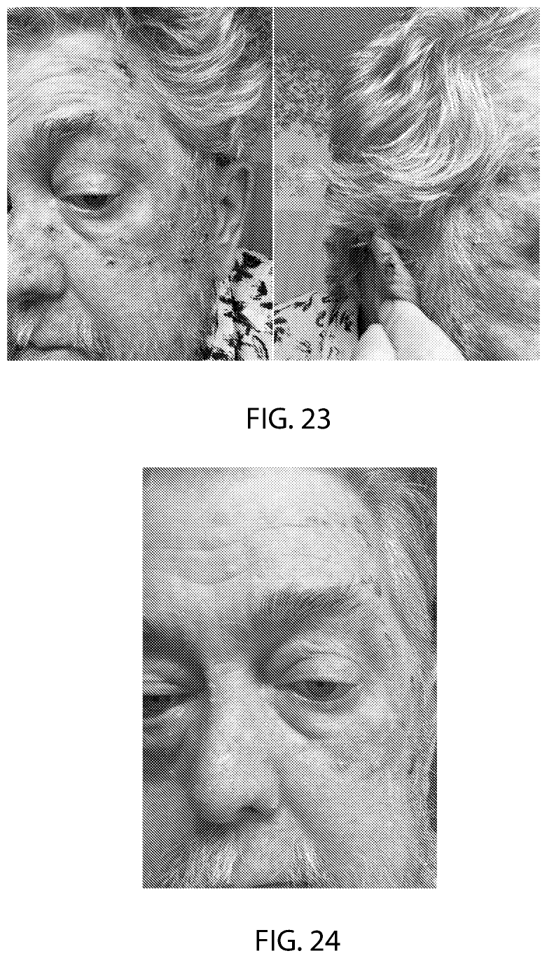

Photos of the patient were taken during the course of the treatment, some of which are included in FIGS. 23-24.

### CASE Study: Individual 1

A 63-year-old male with extensive basil cell carcinoma lesions on both sides of the face, forehead and ear.
Note: the clinical study involving Individual 1, due to limitations in available material, at the time was performed with very low concentrations of W1469 and the patient was instructed to use only on the nose area and under the area of the left cheek. The treatment was thus self-administered. A photo of the individual before treatment is included in FIG. 23. Patient condition was monitored and photographed regularly during the treatment period. Treatment was given throughout a three week period including certain days where treatment was applied at a higher concentration): As shown in FIG. 24, at the end of the treatment period, the treatment achieved significant amelioration of the patient's condition and did not result in scarring or other disfigurement as is often the case with other treatments.

### Single Patient Clinical Study Design: Individual 2

Self-administered, variable treatment protocol, drug concentrations and vehicle carrier.
A 65-year-old male with basal cell carcinoma 6 mm×3.5 mm on right check area, and the center of the lesion had a 1.5 circumscribed depression with necrotic tissue and hyperkeratosis. The lesion was tender to palpation especially to the center and the area around the lesion was with hyperpigmentation.
Following the first week of treatment, a marked improvement in the lesion was obtained with almost complete resolution. No necrotic or hyperkeratotic areas were noted. Area is pain free and hyperpigmentation is markedly reduced as well.
After one month following the treatment, another lesion returned to the patient's right cheek displaying same pattern consistent with basal cell carcinoma. Therapy was reintroduced on a three times-daily dosing schedule and with ethanol-based carrier molecule. Lesion displays centralized necrotic tissue and hyperkeratosis.
After two days of treatment the centralized necrotic core came out of the lesion resulting in a bloody clot. The centralized portion of the lesion slowly healed over the next 5-day period.
Continued healing of the centralized portion of the lesion during the 5-day period status post central necrotic tissue extrusion was shown. During this period the lesion began to flatten, the area became pain free, and localized tissue became less erythematic. A bloody clot in central area resolved and crater size decreased. No evidence of centralized necrosis noted and lesion size markedly decreased. Lack of pain and marked reduction in lesion hyperkeratosis was noted as well.

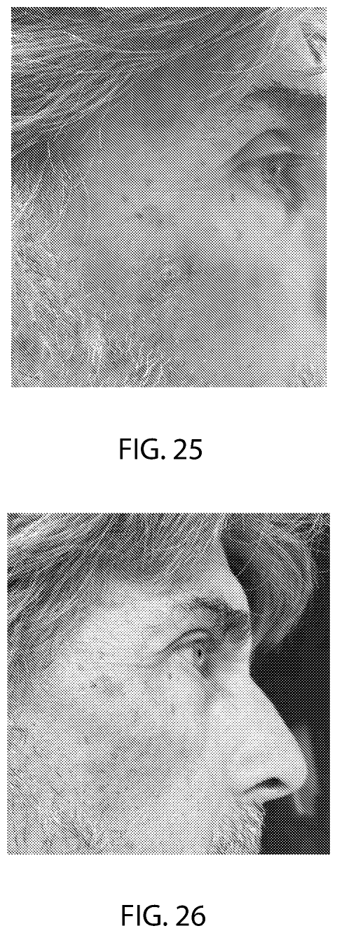

A photo of the individual before treatment is included in FIG. 25. As shown in FIG. 26, significant amelioration of the patient's condition was obtained after 7 days of treatment.

### Case Study: Individual 3

Physician administered, variable treatment protocol, drug concentrations and vehicle carrier.
A 60-year-old female with ulcerative lesion to mid forehead approximately 3 mm×4 mm. Ulcerative lesion to mid forehead approximately 3 mm×4 mm. Non painful and without exudate. Therapy applied on a twice daily dosing schedule. After one week of treatment, there was a resolution of ulcerative lesion noted. No erythema, exudate, or hyperkeratosis noted.

### Case Study: Individual 4

Physician administered, variable treatment protocol, drug concentrations and vehicle carrier.
A 63-year-old male with 2 mm ulcerative lesion (squamous cell carcinoma) to the post-auricular area of left side of the head. The lesion area had been biopsied and treated with cryotherapy one year prior. Lesion area had begun to reappear 3 months prior to this photo. After three days of once daily treatment, the same lesion was already reduced to approximately ½ of original size. Following regular treatment for one week, the affected area had a complete resolution of the patient's lesion.
Note: Subject's initial dosing schedule was once daily. He reported no adverse reactions. Upon next visit (three days after initial dosing) his dose was doubled while maintaining a once daily schedule. Over the next three days his lesion resolved completely and without any adverse reactions noted.

### Case Study Results and Overview

Observations and perceived advantages in all 4 case studies:
EFFICACY (Squamous and Basil Cell):
Progressive reduction of cancer lesion size with signs of possible elimination commensurate with continued administration
Rapid elimination of pain and visible inflammation (2-5 Days)
Marked improvements in general appearance of skin surrounding the cancer lesion(s)
Works at all doses examined. Exhibits dose dependencies: higher concentrations work better.
METHODOLOGY:
Ease of administration. No specialized equipment required.
SAFETY/TOXICITY:
Treatment and application is not painful*
Doesn't inflame or injure adjacent tissue
No side effects were noted

Applications with DMSO as the carrier were briefly painful following administration and discontinued. This was later determined to be the drug carrier and not the drug. Ethanol-based carriers exhibit mild stinging sensation upon application.

In summary, based on the experimental results and the case studies presented above, the compounds of the present disclosure can be used effectively and with fewer side effects in methods of treating cancer and other conditions such as microbial and viral infections, and thus have potential use as promising therapeutics for a broad range of disease indications as described herein.

REFERENCE CITATIONS: The following articles discussed above are incorporated by reference as if set forth in their entirety herein:
Adlard, P. A., Cherny, R. A., Finkelstein, D. I., Gautier, E., Robb, E., Cortes, M., Volitakis, I., Liu, X., Smith, J. P., Perez, K., Laughton, K., Li, Q. X., Charman, S. A., Nicolazzo, J. A., Wilkins, S., Deleva, K., Lynch, T., Kok, G., Ritchie, C. W., Tanzi, R. E., Cappai, R., Masters, C. L., Barnham, K. J., Bush, A. I., 2008. Rapid restoration of cognition in Alzheimer's transgenic mice with 8-hydroxy quinoline analogs is associated with decreased interstitial ap. Neuron 59.
Andersson, D. A., Gentry, C., Moss, S., Bevan, S., 2009. Clioquinol and pyrithione activate TRPA1 by increasing intracellular Zn2ρ. In: Proceedings of the National Academy of Sciences of the United States of America, 106.
Auld, D. S., Kawaguchi, H., Livingston, D. M., Vallee, B. L., 1974. RNA dependent DNA polymerase (reverse transcriptase) from avian myeloblastosis virus: a zinc metalloenzyme. Proc. Natl. Acad. Sci. U.S.A 71, 2091-2095.
Ayton, S., Lei, P., Bush, A. I., 2015. Biometals and their therapeutic implications in Alzheimer's disease. Neurotherapeutics.
Baumgartner M, Sturlan S, Roth E, Wessner B, Bachleitner-Hofmann T. Enhancement of arsenic trioxide-mediated apoptosis using docosahexaenoic acid in arsenic trioxide-resistant solid tumor cells. Int J Cancer 2004; 112:707-12. 27.
Bednarz-Prashad, A. J., John, E. I., 1983. Effect of clioquinol, an 8-hydroxyquinoline derivative, on rotavirus infection in mice. J. Infect. Dis. 148.
Begin M E, Ells G, Das U N, Horrobin D F. Differential killing of human carcinoma cells supplemented with n-3 and n-6 polyunsaturated fatty acids. J Natl Cancer Inst 1986; 77:1053-62.
Bohlmann, L., de Oliveira, D. M. P., E I-Deeb, I. M., Brazel, E. B., Harbison-Price, N., Ong, C. L. Y., Rivera-Hernandez, T., Ferguson, S. A., Cork, A. J., Phan, M. D., Soderholm, A. T., Davies, M. R., Nimmo, G. R., Dougan, G., Schembri, M. A., Cook, G. M., McEwan, A. G., von Itzstein, M., McDevitt, C. A., Walker, M. J., 2018. Chemical synergy between ionophore PBT2 and zinc reverses antibiotic resistance. mBio 9. BPS Bioscience. ACE2 Inhibitor Screening Assay Kit. https://bpsbioscience.com/pub/media/wysiwyg/Proteases/79923_1.pdf.
Bougnoux P. n-3 polyunsaturated fatty acids and cancer. Curr Opin Clin Nutr Metab Care 1999; 2:121-6.
Brewer, G. J., Dick, R. D., Grover, D. K., LeClaire, V., Tseng, M., Wicha, M., Pienta, K., Redman, B. G., Jahan, T., Sondak, V. K. et al. (2000) Treatment of metastatic cancer with tetrathiomolybdate, an anticopper, antiangiogenic agent: Phase I study. Clin. Cancer Res. 6, 1-10
Cahoon, L., 2009. The curious case of clioquinol. Nat. Med. 15.
Cherny, R. A., Ayton, S., Finkelstein, D. I., Bush, A. I., McColl, G., Massa, S. M., 2012. PBT2 reduces toxicity in a C. elegans model of polyQ aggregation and extends lifespan, reduces striatal atrophy and improves motor performance in the R6/2 mouse model of Huntington's disease. J. Huntingt. Dis. 1.
Choi, Y., Bowman, J. W., Jung, J. U., 2018. Autophagy during viral infection—a doubleedged sword. Nat. Rev. Microbiol.
Colvin, R. A., Bush, A. I., Volitakis, I., Fontaine, C. P., Thomas, D., Kikuchi, K., Holmes, W. R., 2008. Insights into Zn2ρ homeostasis in neurons from experimental and modeling studies. Am. J. Physiol. Cell Physiol. 294.
Cox, E. H., McLendon, G. L., 2000. Zinc-dependent protein folding. Curr. Opin. Chem. Biol. Darby, C. M., Nathan, C. F., 2010. Killing of non-replicating Mycobacterium tuberculosis by 8-hydroxyquinoline. J. Antimicrob. Chemother. 65.
Desoize, B. (2003) Metals and metal compounds in carcinogenesis. In Vivo 17, 529-539
Ding, W. Q., Lin, B., Vaught, J. L., Yamauchi, H., Lind, S. E., 2005. Anticancer activity of the antibiotic clioquinol. Canc. Res.
Ding, W. Q., Yu, H. J., Lind, S. E., 2008. Zinc-binding compounds induce cancer cell death via distinct modes of action. Canc. Lett. 271.
Ding, W. Q., Liu, B., Vaught, J. L., Palmiter, R. D., and Lind, S. E., (2006) Clioquinol and docosahexaenoic acid act synergistically to kill tumor cells. Mol. Cancer Ther. 5 1864-1872.
Ding W Q, Vaught J L, Yamauchi H, Lind S E. Differential sensitivity of cancer cells to docosahexaenoic acid-induced cytotoxicity: the potential importance of down-regulation of superoxide dismutase 1 expression. Mol Cancer Ther 2004; 3:1109-17.
Fukunaga K, Hossain Z, Takahashi K: Marine phosphatidylcholine suppresses 1,2-dimethylhydrazine-induced colon carcinogenesis in rats by inducing apoptosis. Nutr Res 2008, 28:635-640.
Ge, E. J, et al., Connecting copper and cancer: from transition metal signaling to metalloplasia, Nat. Rev Cancer 2022 102-113.
Germain E, Chajes V, Cognault S, Lhuillery C, Bougnoux P. Enhancement of doxorubicin cytotoxicity by polyunsaturated fatty acids in the human breast tumor cell line MDA-MB-231: relationship to lipid peroxidation. Int J Cancer 1998; 75:578-83.
Giglione, C., Vallon, O., Meinnel, T., 2003. Control of protein lifespan by N-terminal methionine excision. EMBO J. 22.
Gupta et al., “Insights of 8-hydroxyquinolines: a novel target in medicinal chemistry,” Bioorganic chemistry 108 (2021) 104633
A. Gupte and R. J. Mumper, “Elevated copper and oxidative stress in cancer cells as a target for cancer treatment,” Cancer Treat. Rev., 2009, 35, 32
Haase, H., Overbeck, S., Rink, L., 2008. Zinc supplementation for the treatment or prevention of disease: current status and future perspectives. Exp. Gerontol.
Hanaa H. Abd El Baky et al Der Pharma Chemica, 2014, 6 (5):370-382
Heiger. I, (1926) The Effect of Copper Compounds upon the growth of Carcinoma in the Rat, Biochem J20(2):232-236 doi: 10.1042/bj0200232
Hordyjewska, A., Popiotek, L., and Kocot, J., “The many “faces” of copper in medicine and treatment,” Biometals (2014) 27:611-624
Kafrawy, O., et al., “Docosahexaenoic acid in phosphatidylcholine mediates cytotoxicity more effectively than other w-3 and w-6 fatty acids,” Cancer Letters 132 (1998) 23-29
Kahlson, M., Dixon, S. J., 2022. Copper-induced cell death, vol 375, 1231-1232. And the main paper, cite it same issue.
Kono, R., 1971. Subacute myelo-optico-neuropathy, a new neurological disease prevailing in Japan. Jpn. J. Med. Sci. Biol. 24.
Lannfelt, L., Blennow, K., Zetterberg, H., Batsman, S., Ames, D., Harrison, J., Masters, C. L., Targum, S., Bush, A. I., Murdoch, R., Wilson, J., 2008. Safety, efficacy, and biomarker findings of PBT2 in targeting Abeta as a modifying therapy for Alzheimer's disease: a phase IIa, double-blind, randomised, placebo-controlled trial. Lancet Neurol.
Lind, S. E., Park, J. S., Drexler, J. W., 2009. Pyrithione and 8-hydroxyquinolines transport lead across erythrocyte membranes. Transl. Res. 154.
McInerney, M. P., Volitakis, I., Bush, A. I., Banks, W. A., Short, J. L., Nicolazzo, J. A., 2018. Ionophore and biometal modulation of P-glycoprotein expression and function in human brain microvascular endothelial cells. Pharmaceut. Res. 35.
Meade, T. W., 1975. Subacute myelo optic neuropathy and clioquinol. An epidemiological case history for diagnosis. Br. J. Prev. Soc. Med.
Millet, J. K., Whittaker, G. R., 2015. Host cell proteases: critical determinants of coronavirus tropism and pathogenesis. Virus Res. 202.
Monson, et. al., 1991 Tumor targeted delivery of 8-hydroxyquinoline, Int. J. Radiation Oncology Biol. Phys. Vol 20, 1263-1271.
Olaleye, O., Raghunand, T. R., Bhat, S., Chong, C., Gu, P., Zhou, J., Zhang, Y., Bishai, W. R., Liu, J. O., 2011. Characterization of clioquinol and analogues as novel inhibitors of methionine aminopeptidases from Mycobacterium tuberculosis. Tuberculosis, 91.
Oliveri, V, Giuffrida, M. L., Vecchio, G., Aiello, C., Viale, M. “Glucoconjugates of 8-hydroxyquinolines as potential anti-cancer prodrugs,” Dalton Trans., 2012, 41, 4530.
Oliveri, V. and Vecchio, G., Iyne “Glycoconjugates of Quinolines: Application in Medicinal Chemistry,” Mini-Reviews in Medicinal Chemistry, 2016, 16, 1185-1194
Oliveri, V. Front Mol Biosci 2022 Mar. 4; 9: 84184. review “Selective Targeting of Cancer Cells by Copper Ionophores: An Overview”.
Omonike A. Olaleye *, Manvir Kaur, Collins Onyenaka, Tolulope Adebusuyi, Discovery of Clioquinol and analogues as novel inhibitors of Severe Acute Respiratory Syndrome Coronavirus 2 infection, ACE2 and ACE2—Spike protein interaction in vitro Heliyon 7 (2021) e06426
Patterson, J. B, Putz, A. T., Tao, L., Simke, W. C., Bryant III, H. B., Britt, R. D., Li, B., “Biosynthesis of fluopsin C, a copper-containing antibiotic from Pseudomonas aeruginosa,” 2021, Science 374, 1005-1009.
Perez, D. R., Sklar, L. A., Chigaev, A., 2019. Clioquinol: to harm or heal. Pharmacol. Ther.
Huang, R., Wallqvist, A. and Covell, D. G. (2005) Anticancer metal compounds in NCI's tumor-screening database: putative mode of action. Biochem. Pharmacol. 69, 1009-1039
Ritchie, C. W., Bush, A. I., Mackinnon, A., Macfarlane, S., Mastwyk, M., MacGregor, L., Kiers, L., Cherny, R., Li, Q. X., Tammer, A., Carrington, D., Mavros, C., Volitakis, I., Xilinas, M., Ames, D., Davis, S., Beyreuther, K., Tanzi, R. E., Masters, C. L., 2003. Metal-protein attenuation with iodochlorhydroxyquin (clioquinol) targeting aβ amyloid deposition and toxicity in alzheimer disease: a pilot phase 2 clinical trial. Arch. Neurol. 60.
Roden, D. M., Harrington, R. A., Poppas, A., Russo, A. M., 2020. Considerations for drug interactions on QTc interval in exploratory COVID-19 treatment. J. Am. Coll. Cardiol. 75, 2623 LP-2624.
Rohini Gupta, Vijay Luxami, Kamaldeep Paul “Insights of 8-hydroxyquinolines: A novel target in medicinal chemistry,”, Bioinorganic Chemistry: 108, 104633 (2021)
Sanders, J. M., Monogue, M. L., Jodlowski, T. Z., Cutrell, J. B., 2020. Pharmacologic treatments for coronavirus disease 2019 (COVID-19): a review. J. Am. Med. Assoc.
Schimmer, A. D., Jitkova, Y., Gronda, M., Wang, Z., Brandwein, J., Chen, C., Gupta, V., Schuh, A., Yee, K., Chen, J., Ackloo, S., Booth, T., Keays, S., Minden, M. D., 2012, “A phase I study of the metal ionophore clioquinol in patients with advanced hematologic malignancies”, Clin. Lymphoma, Myeloma & Leukemia 12.
Shi, L., Huang, C., Luo, Q., Xia, Y., Liu, W., Zeng, W., Cheng, A., Shi, R., Zhengli, C., 2020. Clioquinol improves motor and non-motor deficits in MPTP-induced monkey model of Parkinson's disease through AKT/mTOR pathway. Aging 12.
Tavares, G. de S. V., Mendonga, D. V. C., Lage, D. P., Granato, J. da T., Ottoni, F. M., Ludolf, F., Chavez-Fumagalli, M. A., Duarte, M. C., Tavares, C. A. P., Alves, R. J., Coimbra, E. S., Coelho, E. A. F., 2018. Antileishmanial activity, cytotoxicity and mechanism of action of clioquinol against Leishmania infantum and Leishmania amazonensis species. Basic Clin. Pharmacol. Toxicol. 123.
Tavares, G. S. V., Mendonga, D. V. C., Pereira, I. A. G., Oliveira-Da-Silva, J. A., Ramos, F. F., Lage, D. P., MacHado, A. S., Carvalho, L. M., Reis, T. A. R., Perin, L., Carvalho, A. M. R. S., Ottoni, F. M., Ludolf, F., Freitas, C. S., Bandeira, R. S., Silva, A. M., Chavez Fumagalli, M. A., Duarte, M. C., Menezes-Souza, D., Alves, R. J., Roatt, B. M., Coelho, E. A. F., 2020. A clioquinol-containing Pluronic® F127 polymeric micelle system is effective in the treatment of visceral leishmaniasis in a murine model. Parasite 27.
Toyokuni, S. (1996) Iron-induced carcinogenesis: the role of redox regulation. Free Radical Biol. Med. 20, 553-566
Theophanides, T. and Anastassopoulou, J. (2002) Copper and carcinogenesis. Crit. Rev. Oncol. Hematol. 42, 57-64 4
Valentina Oliveri,a Maria Laura Giuffrida,b Graziella Vecchio,*a Cinzia Aielloc and Maurizio Viale*, “Gluconjugates of 8-hydroxyquinolines as potential anti-cancer prodrugs.” Dalton Trans., 2012, 41, 4530
Vander Heiden, M. G., Cantley, L. C., and Thompson, C. B. Understanding the Warburg Effect: The Metabolic Requirements of Cell Proliferation, Science 2009 324(5930) 1-29-1033
Wei-Qun Ding, Bolin Liu, Joshua L. Vaught, Richard D. Palmiter, and Stuart E. Lind, Clioquinol and docosahexaenoic acid act synergistically to kill tumor cells, Mol. Cancer Ther. 2006, 5:1864-1872.
Xu, H., et al., “8-hydroxyquinoline\ddot{ }a privileged structure with broad-ranging pharmacological potential,” Med Ghem Comm, 2015 (anti-HIV discussed).
Yesilagac, et. al., Enzymatic synthesis of ^{125/131I} I labeled 8-hydroxyquinoline glucuronide and in vitro/in vivo evaluation of biological influence, Applied Radiation and Isotopes, 69, (2011) 299-307
You, Z., Ran, X., Dai, Y., Ran, Y., 2018. Clioquinol, an alternative antimicrobial agent against common pathogenic microbe. J. Mycol. Med. 28.
Yu, H., Zhou, Y., Lind, S. E., Ding, W. Q., 2009. Clioquinol targets zinc to lysosomes in human cancer cells. Biochem. J. 417.
Yu et al., Clioquinol targets zinc to lysosomes in human cancer cells, Biochem. J. (2009) 417, 133-139 (Printed in Great Britain) doi:10.1042/BJ20081421
Yueying Zhen, Kristopher W. Krausz, Chi Chen, Jeffrey R. Idle, and Frank J. Gonzalez, Metabolomic and Genetic Analysis of Biomarkers for PPARα Expression and Activation, Mol Endocrinol. 2007 September; 21(9): 2136-2151.
Zhang, M., Xie, M., Li, S., Gao, Y., Xue, S., Huang, H., Chen, K., Liu, F., Chen, L., 2017. Electrophysiologic studies on the risks and potential mechanism underlying the proarrhythmic nature of azithromycin. Cardiovasc. Toxicol. 17, 434-440.
Zhai, S., “Tumor cellular proteasome inhibition and growth suppression by 8-hydroxyquinoline and clioquinol requires their capabilities to bind copper and . . . ”, JBIC Journal of Biological . . . 2010 Springer

1. A pharmaceutical composition comprising 8-Hydroxy-2-quinolinyl beta-D-glucopyranosiduronic acid (G2-8HQ) and a pharmaceutically acceptable vehicle, excipient, or carrier.

2-9. (canceled)

10. The pharmaceutical composition of claim 1 wherein the 8-Hydroxy-2-quinolinyl beta-D-glucopyranosiduronic acid is at a concentration in the range of about 100 μM to about 500 mM.

11. The pharmaceutical composition of claim 1 wherein the 8-Hydroxy-2-quinolinyl beta-D-glucopyranosiduronic acid is at a concentration in the range of about 300 μM to about 150 mM.

12. The pharmaceutical composition of claim 1 wherein the 8-Hydroxy-2-quinolinyl beta-D-glucopyranosiduronic acid is at a concentration in the range of about 1 mM to about 50 mM.

13. The pharmaceutical composition of claim 1 wherein the 8-Hydroxy-2-quinolinyl beta-D-glucopyranosiduronic acid is at a concentration in the range of about 1 mM to about 25 mM.

14-15. (canceled)

16. A method of treating cancer comprising administering an effective amount of the composition of claim 1 to a subject in need thereof.

17. The method of claim 16 wherein the composition is administered in a form selected from the group consisting of oral administration, injection, intravenous (IV), suppository, topical, and spray/mist.

18-22. (canceled)

23. A method of treating a condition selected from the group consisting of viral infection, a microbial infection, asthma, neurodegeneration, and inflammation comprising administering an effective amount of the composition of claim 1 to a subject in need thereof.

24-26. (canceled)

27. The method of claim 23 wherein the neurodegeneration is selected from the group consisting of Alzheimer's disease, multiple sclerosis (MS), amyotrophic lateral sclerosis (ALS), and Parkinson's disease.

28-77. (canceled)

78. A pharmaceutical composition comprising a glucuronic acid derivative selected from the group consisting of 8-Hydroxy-2-quinolinyl beta-D-glucopyranosiduronic acid, 8-Hydroxy-3-quinolinyl beta-D-glucopyranosiduronic acid, 8-Hydroxy-4-quinolinyl beta-D-glucopyranosiduronic acid, 8-Hydroxy-5-quinolinyl beta-D-glucopyranosiduronic acid, 8-Hydroxy-6-quinolinyl beta-D-glucopyranosiduronic acid, 8-Hydroxy-7-quinolinyl beta-D-glucopyranosiduronic acid, 2-beta-D-glucopyranosiduronic acid-5-chloro-7-iodo-8-hydroxyquinoline; 8-Hydroxy-2-quinolinyl beta-D-glucopyranoside, 8-Hydroxy-3-quinolinyl beta-D-glucopyranoside. 8-Hydroxy-4-quinolinyl beta-D-glucopyranoside, 8-Hydroxy-5-quinolinyl beta-D-glucopyranoside, 8-Hydroxy-6-quinolinyl beta-D-glucopyranoside, 8-Hydroxy-7-quinolinyl beta-D-glucopyranoside, and 2-beta-D-glucopyranoside-5-chloro-7-iodo-8-hydroxyquinoline, and a pharmaceutically acceptable vehicle, excipient, or carrier.

79-85. (canceled)

86. A pharmaceutical composition comprising a 2,8-dihydroxy quinolone glucuronic acid derivative and a pharmaceutically acceptable vehicle, carrier, or excipient.

87. The pharmaceutical composition according to claim 86 wherein the glucuronic acid derivative is 8-Hydroxy-2-quinolinyl beta-D-glucopyranosiduronic acid.

88. (canceled)

89. A method of preparing the composition of claim 1 comprising isolating G2-8HQ from the serum of an ungulate and combining G2-8HQ with a pharmaceutically acceptable vehicle, carrier, or excipient.

90. The method of claim 89 wherein the ungulate is a goat.

91. A method for use in treating cancer comprising administering to a subject in need thereof an effective amount of a pharmaceutical composition comprising 8-Hydroxy-2-quinolinyl beta-D-glucopyranosiduronic acid (G2-8HQ) and a pharmaceutically acceptable vehicle, excipient, or carrier.

92-95. (canceled)

96. A pharmaceutical composition for use in treating cancer comprising 8-Hydroxy-2-quinolinyl beta-D-glucopyranosiduronic acid (G2-8HQ) and a pharmaceutically acceptable vehicle, excipient, or carrier:

97-98. (canceled)

99. Use of an isolated 8-Hydroxy-2-quinolinyl beta-D-glucopyranosiduronic acid for treatment of cancer in a human or animal subject in need thereof.

100. The use of claim 99 wherein the isolated 8-Hydroxy-2-quinolinyl beta-D-glucopyranosiduronic acid is used with a pharmaceutically acceptable vehicle, carrier, or excipient.

101. Use of an isolated 8-Hydroxy-2-quinolinyl beta-D-glucopyranosiduronic acid for treatment of a condition selected from the group consisting of a viral infection, a microbial infection, a parasitic infection, asthma, neurodegeneration, and inflammation.

102. The use of claim 101 wherein the isolated 8-Hydroxy-2-quinolinyl beta-D-glucopyranosiduronic acid is used with a pharmaceutically acceptable vehicle, carrier, or excipient.

103-109. (canceled)
